(19) United States
(12) Patent Application Publication
(10) Pub. No.: US 2016/0303232 A1
(43) Pub. Date: Oct. 20, 2016

(54) COMBINATION TREATMENTS WITH SERIBANTUMAB

(71) Applicant: Merrimack Pharmaceuticals, Inc., Cambridge, MA (US)

(72) Inventors: Bambang ADIWIJAYA, Belmont, MA (US); Akos Czibere, Medford, MA (US); Rachel C. Nering, Stoneham, MA (US); Gavin Macbeath, Wakefield, MA (US)

(21) Appl. No.: 15/156,603

(22) Filed: May 17, 2016

Related U.S. Application Data
(63) Continuation-in-part of application No. PCT/US2016/027933, filed on Apr. 15, 2016.
(60) Provisional application No. 62/149,271, filed on Apr. 17, 2015.

(51) Int. Cl. A61K 39/395; A61K 31/519; C07K 16/28; A61K 31/337; A61K 9/00

(52) U.S. Cl. CPC A61K 39/39558; A61K 31/337; A61K 9/0019; C07K 16/2863; A61K 31/519; A61K 2039/505

(57) ABSTRACT

Compositions and methods for treating a cancer in a selected human patient are provided, comprising administering to the patient a combination of an anti-ErbB3 antibody (e.g., Seribantumab) and a second anti-cancer therapeutic. A cancer to be treated by the methods and compositions disclosed herein includes cancers that are heregulin (HRG) positive cancers.

### RELATED APPLICATIONS

This application is a continuation-in-part of International Application No. PCT/US2016/027933 (filed Apr. 15, 2016), which claims priority to, and the benefit of, U.S. Provisional Application No. 62/149,271 (filed Apr. 17, 2015). The contents of the aforementioned applications are hereby incorporated by reference in their entireties.

### SEQUENCE LISTING

The instant application contains a Sequence Listing which has been submitted electronically in ASCII format and is hereby incorporated by reference in its entirety. Said ASCII copy, created on May 17, 2016, is named MMJ_053PCCP_SL.txt and is 21,625 bytes in size.

### BACKGROUND

### Non-Small-Cell Lung Cancer (NSCLC)

Lung cancer is one the leading causes of cancer-related deaths worldwide. There were estimated to be 224,410 new cases diagnosed in 2014 alone, making up approximately 13% of all cancer diagnoses. For cases diagnosed during the period of 2003-2009, the 1- and 5-year survival rates were 43% and 17% respectively (“American Cancer Society Facts and Figures 2014”). Over 80% of lung cancers are non-small cell lung cancers (NSCLC), and nearly two thirds of these are diagnosed at an advanced stage. A platinum-based doublet regimen with a “third-generation” agent (paclitaxel, docetaxel, gemcitabine, vinorelbine, or pemetrexed) is considered standard of care worldwide for the treatment of advanced NSCLC. However, only one third of patients that receive this regimen reach an objective response during first-line therapy, and another 20-30% achieves stabilization of disease. Unfortunately, almost all such patients ultimately see progression of their disease.

### Current Treatments for NSCLC

Three agents that are currently approved for treatment of refractory (recurrent, i.e., second-line treatment) advanced NSCLC are docetaxel, pemetrexed, and erlotinib.

Docetaxel, brand names TAXOTERE®, DOCECAD®—IUPAC name 1,7β,10β-trihydroxy-9-oxo-5β,20-epoxytax-11-ene-2α,4,13α-triyl 4-acetate 2-benzoate 13-{(2R,3S)-3-[(tert-butoxycarbonyl)amino]-2-hydroxy-3-phenylpropanoate}, is an anti-mitotic taxane anti-cancer therapeutic that is typically administered via a one-hour infusion every three weeks over ten or more cycles. The approved dose of docetaxel in the second-line treatment of NSCLC is 75 mg/m² intravenously over 60 minutes once every 3 weeks. Docetaxel should be administered prior to seribantumab dosing.

Pemetrexed, brand name ALIMTA®—IUPAC name (2S)-2-{[4-[2-(2-amino-4-oxo-1,7-dihydro pyrrolo[2,3-d]pyrimidin-5-yl)ethyl]benzoyl]amino}pentanedioic acid), is a folate antimetabolite currently approved for the treatment of pleural mesothelioma and non-small cell lung cancer. It is typically administered at a dose of 500 mg/m² intravenously over 10 minutes on day 1 of each 21-day cycle.

### Ovarian Cancer

Ovarian cancer, including epithelial ovarian cancer is a leading cause of cancer-related death in women, as are primary peritoneal carcinoma and fallopian tube carcinoma. Since ovarian cancer is relatively asymptomatic at its early stages, it often remains undiagnosed until the disease has reached an advanced stage. The standard treatment for advanced ovarian cancer includes surgery followed by chemotherapy with a platinum-based chemotherapeutic agent, e.g., cisplatin, carboplatin, oxaliplatin, and satraplatin, or with an antimicrotubule agent such as paclitaxel. Other drugs used to treat ovarian cancer include bevacizumab, carboplatin, cyclophosphamide, doxorubicin, gemcitabine, olaparib, and topotecan. Although standard treatments are often successful, many patients suffer a recurrence of the disease, often with expression of resistance to platinum-based regimens.

Seribantumab, an anti-ErbB3 Monoclonal Antibody Therapeutic

Seribantumab (previously MM-121 or Ab #6) is an human monoclonal anti-ErbB3 IgG2; see, e.g., U.S. Pat. Nos. 7,846,440; 8,691,771 and 8,961,966; 8,895,001, U.S. Patent Publication Nos., 20110027291, 20140127238, 20140134170, and 20140248280), as well as international Publication Nos. WO/2013/023043, WO/2013/138371, WO/2012/103341, and U.S. patent application Ser. No. 14/967,158.

Seribantumab is a recombinant human IgG2 mAb that binds an epitope on human ErbB3 with high specificity. The complete tetrameric structure of the IgG2 molecule is composed of 2 heavy chains (445 amino acids each) and 2 lambda light chains (217 amino acids each) held together by intrachain and interchain disulfide bonds. The amino acid sequence (see below) predicts a molecular weight of 143 kDa for the intact nonglycosylated monomer IgG2. Glycosylation analysis demonstrates N-linked glycosylation of seribantumab, which is predicted to contribute approximately 2.9 kDa to the molecular weight of the intact glycosylated seribantumab monomer. The predicted molecular weight of intact glycosylated seribantumab, 146 kDa, is within 0.2% of the actual molecular weight as experimentally determined by mass spectroscopy. The isolectric point of seribantumab is approximately 8.6 (major isoform as determined by isoelectric focusing electrophoresis).

Seribantumab is administered by intravenous infusion (e.g., over the course of one hour) and is supplied as a clear liquid solution in sterile, single-use vials containing 10.1 ml of seribantumab at a concentration of 25 mg/ml in an aqueous solution of 20mM histidine, 150mM sodium chloride, at a pH of about 6.5 (in the range of 6.2 to 6.8), to be stored at 2-8° C. Seribantumab comprises a heavy chain having the amino acid sequence of SEQ ID NO:7 and a light chain having the amino acid sequence of SEQ ID NO:8. Seribantumab comprises a heavy chain variable region (VH) and a light chain variable region (VL) encoded by the nucleic acid sequences set forth in SEQ ID NOs:9 and 11, respectively. Seribantumab comprises VH and VL regions comprising the amino acid sequences set forth in SEQ ID NOs:10 and 12, respectively. Seribantumab comprises CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3).

### Evaluation of Treatment Outcomes

Treatment outcomes for NSCLC, ovarian cancer, primary peritoneal carcinoma and fallopian tube carcinoma are evaluated using standard measures for tumor response.
TARGET LESION (tumor) responses to therapy are classified as:

Complete Response (CR): Disappearance of all target lesions. Any pathological lymph nodes (whether target or non-target) must have reduction in short axis to <10 mm; Partial Response (PR): At least a 30% decrease in the sum of the diameters of target lesions, taking as reference the baseline sum diameters;

Progressive Disease (PD: At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and

Stable Disease (SD): Neither sufficient shrinkage to qualify for PR nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.
NON-TARGET LESION responses to therapy are classified as:

Complete Response (CR): Disappearance of all non-target lesions and normalization of tumor marker levels. All lymph nodes must be non-pathological in size (<10 mm short axis). If tumor markers are initially above the upper normal limit, they must normalize for a patient to be considered in complete clinical response;

Non-CR/Non-PD: Persistence of one or more non-target lesion(s) and/or maintenance of tumor marker level above the normal limits; and

Progressive Disease (PD): Either or both of appearance of one or more new lesions and unequivocal progression of existing non-target lesions. In this context, unequivocal progression must be representative of overall disease status change, not a single lesion increase.

Other Exemplary Positive Responses

Patients treated with these methods may experience improvement in at least one sign of NSCLC or ovarian cancer, primary peritoneal carcinoma and fallopian tube carcinoma. Response may also be measured by a reduction in the quantity and/or size of measurable tumor lesions. Measurable lesions are defined as those that can be accurately measured in at least one dimension (longest diameter is to be recorded) as >10 mm by CT scan (CT scan slice thickness no greater than 5 mm), 10 mm caliper measurement by clinical exam or >20 mm by chest X-ray. The size of non-target lesions, e.g., pathological lymph nodes can also be measured for improvement. Lesions can be measured using, e.g., x-ray, CT, or MM images. Microscopy, cytology or histology can be also used to evaluate responsiveness to a therapy. An effusion that appears or worsens during treatment when a measurable tumor has otherwise met criteria for response or stable disease can be considered to indicate tumor progression, but only if there is cytological confirmation of the neoplastic origin of the effusion.

Although the currently approved treatments for NSCLC ovarian cancer, primary peritoneal carcinoma and fallopian tube carcinoma provide some benefit, there is still much room for improvement, particularly for patients with advanced or metastatic disease. Thus more effective treatments for patients with advanced NSCLC, ovarian cancer, primary peritoneal carcinoma and fallopian tube carcinoma are needed. The present invention addresses this need and provides additional benefits.

### SUMMARY

Provided are compositions and methods for treating a cancer in a selected human patient, comprising administering to the patient a combination of an anti-ErbB3 antibody and a second anti-cancer therapeutic.

The cancer may be a non-small cell lung cancer (NSCLC) e.g., nonsquamous NSCLC, and the second anti-cancer therapeutic may be, e.g., docetaxel or pemetrexed, wherein the combination is administered (or is for administration) according to a particular clinical dosage regimen (i.e., at a particular dose amount and according to a specific dosing schedule). The cancer may instead be an ovarian cancer (e.g., persistent, recurrent, resistant, or refractory ovarian cancer) or the cancer may be primary peritoneal carcinoma or fallopian tube carcinoma and, for each of these the second anti-cancer therapeutic may be, e.g., paclitaxel, gemcitabine, irinotecan, liposomal irinotecan (e.g., nal-IRI) or liposomal doxorubicin, e.g., DOXIL®. In one embodiment, the cancer is a locally advanced or metastatic NSCLC that has progressed (i.e., is treatment refractory) after prior therapy with an organoplatinum agent. In one embodiment, the NSCLC is squamous cell carcinoma. In another embodiment, the cancer is EGFR wild-type.

In one aspect, a method of treating a cancer in an adult human patient is provided, the method comprising administering to the patient an anti-ErbB3 antibody comprising CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3), wherein the anti-ErbB3 antibody is administered as a first single dose of 3000 mg, regardless of patient body mass. In one embodiment, the first single dose is followed by at least one additional single dose, each of which at least one additional dose is administered three weeks after the immediately prior dose and is administered at a dosage of 3000 mg, regardless of patient body mass.

In a second aspect a method of treating a cancer patient who has a NSCLC tumor; and has progressed following treatment with no more than two systemic therapies for locally advanced or metastatic disease, of which one if which therapies was a platinum-based regimen is provided; the method comprising administering to the patient an effective amount of each of (1) an anti-ErbB3 antibody comprising CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3), and (2) docetaxel or pemetrexed.

In a third aspect a composition for treating a cancer in an adult human patient is provided, the composition comprising an antibody comprising CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3), wherein the composition is for administration as a first single dose of 3000 mg, regardless of patient body mass. In one embodiment, the composition is for administration as a first single dose of 3000 mg, regardless of patient body mass, followed by at least one additional single dose, each of which at least one additional dose is administered three weeks after the immediately prior dose and is administered at a dosage of 3000 mg, regardless of patient body mass.

In one embodiment, the cancer is non-small cell lung cancer (NSCLC). In another embodiment, the cancer is ovarian cancer.

In one embodiment, the patient has progressed following treatment with no more than two systemic therapies for locally advanced or metastatic disease, of which one was a prior platinum-based regimen. In another embodiment, the patient has progressed following treatment with no more than three systemic therapies for locally advanced or metastatic disease, of which one was a prior platinum-based regimen. In another embodiment, the human patient is treated following disease progression or recurrence after prior treatment with antineoplastic therapy (e.g., anti-cancer agent). In another embodiment, the human patient is treated after failure of an antineoplastic therapy. In another embodiment, the cancer is identified as a cancer that has acquired resistance to antineoplastic therapy.

In exemplary embodiments of any of the above aspects, the methods disclosed herein further comprise coadministration of an effective amount of a second anti-cancer therapeutic with the anti-ErbB3 antibody. In one embodiment, the second anti-cancer therapeutic is docetaxel, and wherein the effective amount of docetaxel is 75 mg/m². In another embodiment the second anti-cancer therapeutic is pemetrexed, and wherein the effective amount is 500 mg/m². In one embodiment, the effective amount of the docetaxel or pemetrexed is co-administered at least 30 minutes before the administration of the antibody.

In a fourth aspect, a composition for treating a cancer in an adult human patient is provided, the composition comprising an antibody comprising CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3), wherein the composition is for administration as a first single dose of 3000 mg, regardless of patient body mass. In one embodiment, the composition is for administration as a first single dose of 3000 mg, regardless of patient body mass, followed by at least one additional single dose, each of which at least one additional dose is administered three weeks after the immediately prior dose and is administered at a dosage of 3000 mg, regardless of patient body mass. In another embodiment, the composition is for administration at a dose of 20 mg/kg. In one embodiment, the ovarian cancer is persistent, recurrent, resistant, or refractory ovarian cancer.

In a fifth aspect, a method of treating a cancer patient who has an ovarian tumor is provided, a primary peritoneal carcinoma or a fallopian tube carcinoma, the method comprising administering to the patient an effective amount of each of (1) an anti-ErbB3 antibody comprising CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3), and (2) paclitaxel, irinotecan, or gemcitabine.

In exemplary embodiments of any of the above aspects, the anti-ErbB3 antibody is seribantumab.

In one embodiment the treatment methods described herein comprise administering seribantumab in combination with one or more other antineoplastic agents (e.g., other chemotherapeutics, other anti-cancer agents, or other small molecule drugs).

In one embodiment, no more than three other anti-cancer therapeutics are administered within a treatment cycle. In another embodiment, no more than two other anti-cancer therapeutics are administered in combination with seribantumab within the treatment cycle. In another embodiment, no more than one other anti-cancer therapeutic is administered in combination with seribantumab within the treatment cycle. In another embodiment, no other anti-cancer therapeutic is administered in combination with seribantumab within the treatment cycle. In another embodiment, the other anti-cancer therapeutics may be administered either simultaneously or before or after administration of seribantumab.

A cancer to be treated by the methods and compositions disclosed herein includes cancers that are heregulin (HRG) positive cancers, optionally wherein HRG positivity is determined by a HRG RNA-ISH assay or a quantitative RT-PCR assay. In such assay a sample is determined to be positive if such assay reveals at least 1-3 dots per cell, wherein the cells are from patient tumor samples. In one embodiment, HRG positivity is based on an FDA-approved test. In one embodiment, the cancer is non-small cell lung cancer (NSCLC). In another embodiment, the cancer is locally advanced or metastatic. In another embodiment, the patient has progressed following treatment with no more than two systemic therapies for locally advanced or metastatic disease, one of which systemic therapies comprised a platinum-based regimen.

In one embodiment, the treatment of a cancer comprising the compositions and/or methods of any of the above aspects produces at least one therapeutic effect selected from the group consisting of: reduction in size of a tumor, reduction in metastasis, complete remission, partial remission, stable disease, increase in overall response rate, or a pathologic complete response.

### BRIEF DESCRIPTION OF THE DRAWINGS

FIG. 1 shows that the capacity of heregulin (HRG) to induce proliferation in a panel of NSCLC cell lines in vitro is indicative of single-agent response to seribantumab in vivo. Nine out of 25 EGFR wild-type NSCLC cell lines are responsive to HRG; they exhibit increased cell proliferation in response to exogenously added HRG, as measured by CellTiter-Glo® (CTG) using 3D spheroid cultures.

FIGS. 2A-2D are four graphs showing that cells responsive to HRG in vitro responded to seribantumab in vivo, while cell lines not responsive to HRG in vitro did not respond to seribantumab in vivo. HRG-responsive cell lines A549 (FIG. 2A) and H322M (FIG. 2B) as well as HRG non-responsive cell lines H460 (FIG. 2C) and HOP-92 (FIG. 2D) are shown. Tumor volume over time is shown as indicative of seribantumab response.

FIGS. 3A-3D are four graphs showing that 5 nM HRG induces resistance to docetaxel (111 nM, FIG. 3A) and pemetrexed (1111 nM, FIG. 3B) in a 3D spheroid proliferation assay in multiple cell lines after 96hrs; FIG. 3C and FIG. 3D show that treatment with seribantumab (1 μM, “MM-121”) restores sensitivity to docetaxel (FIG. 3C) and pemetrexed (FIG. 3D) in NSCLC cell lines (A549, EKVX, H358, H322M, Calu-3, H661, H441, H1355, H430).

FIG. 4 is a set of graphs showing HRG mRNA expression levels across different indications based on the TCGA data set.

FIGS. 5A and 5B are two graphs shows HRG mRNA expression across NSCLC tissue samples from both the MM-121-01-101 phase II Study (FIG. 5A) and commercially-sourced biopsy specimens (FIG. 5B).

FIGS. 6A-6C are a set of box and whisker plots (indicating interquartile ranges and outliers) showing seribantumab pharmacokinetics for weight-based and fixed dosing regimens by doses and intervals. FIG. 6A shows seribantumab maximum concentration (Cmax, mg/L), FIG. 6B shows seribantumab minimum concentration (Cmin, mg/L), and FIG. 6C shows seribantumab average concentration (AvgConc, mg/L). Weight-based and fixed doses are indicated along the y-axis.

FIGS. 7A-7C are a set of graphs showing that heregulin mediates resistance to treatment regardless of the class of chemotherapy, and that co-administration with seribantumab (“MM-121”) abrogates this resistance. In a mouse OVCAR8 xenograft model of ovarian cancer, tumor-bearing mice were treated with paclitaxel (FIG. 7A), irinotecan (FIG. 7B), or gemcitabine (FIG. 7C), either alone as monotherapies or with a fixed dose of seribantumab. In each case, the tumors treated with paclitaxel, irinotecan, gemcitabine monotherapy began to progress over time, whereas this effect was greatly reduced when the chemotherapeutics were co-administered with seribantumab. Control mice received PBS alone.

### DETAILED DESCRIPTION

Provided herein are methods for effective treatment of platinum refractory NSCLC (e.g., a locally advanced or metastatic NSCLC) in a human patient using a combination of seribantumab and either a taxane, (e.g., docetaxel) or a folate antimetabolite (e.g., pemetrexed).

### I. Patient Selection

A NSCLC patient selected for treatment is an adult patient who has failed at least one, but not more than three, systemic therapies for locally advanced or metastatic NSCLC, one which failed systemic therapies must have been a platinum-based therapy (e.g., a doublet therapy). In another aspect, the NSCLC patient has one or more NSCLC tumors that are positive for heregulin (HRG) mRNA as assessed by an RNA-ISH assay, as described in the Examples below. In one embodiment, the NSCLC tumor is positive for HRG as assessed by an FDA-approved test.

In another aspect, the invention provides methods for effective treatment of cancer (e.g., NSCLC) in a human patient in need thereof who previously received antineoplastic therapy and developed resistance to the antineoplastic therapy. For example, in one embodiment, the method comprises treating cancer in a human patient in need thereof who previously received antineoplastic therapy and developed resistance to the antineoplastic therapy by administering seribantumab and either a taxane, (e.g., docetaxel) or a folate antimetabolite (e.g., pemetrexed).

### II. Combination Therapies

Seribantumab is to be co-administered with a taxane (e.g., docetaxel) or a folate antimetabolite (e.g., pemetrexed), to a selected subject with NSCLC. In another embodiment, seribantumab is to be co-administered with paclitaxel, irinotecan, or gemcitabine to a selected subject with an ovarian cancer, primary peritoneal carcinoma or fallopian tube carcinoma.

“Co-administer” refers to simultaneous or sequential administration of the seribantumab and the taxane or folate antimetabolite. When sequential, co-administration must occur within a timespan that is short enough so that both the seribantumab and the taxane or folate antimetabolite are simultaneously present in treated patients.

In one embodiment, seribantumab is co-administered with the taxane docetaxel. Docetaxel is approved for single agent use in treating breast cancer and NSCLC (post-platinum therapy), and in combination therapy for treatment of hormone refractory prostate cancer, NSCLC (in combination with cisplatin), gastric adenocarcinoma, and squamous cell carcinoma of the head and neck. The approved dose regimen of docetaxel for the treatment of NSCLC is 75 mg/m², given intravenously over 1 hour, once every 3 weeks.

In another embodiment, seribantumab is co-administered with the folate antimetabolite pemetrexed, also marketed under the trade name ALIMTA®. ALIMTA is approved for combination therapy treatment of non-squamous cell NSCLC and mesothelioma. The recommended dose of ALIMTA is 500 mg/m² i.v. on Day 1 of each 21-day cycle. Dose reductions may be needed if toxicity is observed in combination therapy regimens, and may be adjusted in subsequent cycles.

In another embodiment, no more than three other anti-cancer therapeutics are administered in combination with seribantumab within a treatment cycle. In another embodiment, no more than two other anti-cancer therapeutics are administered in combination with seribantumab within the treatment cycle. In another embodiment, no more than one other anti-cancer therapeutic is administered in combination with seribantumab within the treatment cycle. In another embodiment, no other anti-cancer therapeutic is administered in combination with seribantumab within the treatment cycle. In another embodiment, the other anti-cancer therapeutics may be administered either simultaneously or before or after administration of seribantumab.

As used herein, “antineoplastic agent” refers to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

### III. Treatment Protocols

A selected patient having advanced or metastatic NSCLC is treated on day 1 of at least one 21-day treatment cycle. Prior to the first treatment cycle, the patient undergoes a pre-treatment regimen. The regimen is specific to the upcoming chemotherapeutic treatment (e.g., pemetrexed or docetaxel) and is designed to mitigate pemetrexed- or docetaxel-related toxicity. Docetaxel pre-treatment comprises premedication with a corticosteroid such as dexamethasone (e.g., 8 mg twice daily) for three days, starting one day prior to docetaxel administration. Pemetrexed pre-treatment comprises premedication with a low-dose oral folic acid preparation (or multivitamin containing folic acid) on a daily basis, starting at least seven days before the start of the first 21-day cycle. On day 1 of each 21-day cycle, the patient will receive a standard dose of docetaxel or pemetrexed intravenously at least 30 minutes prior to the administration of seribantumab. Seribantumab is then administered intravenously over 90 minutes (on day 1 of the first 21-day cycle) or 60 minutes (on day 1 of any subsequent 21-day cycle).

As used herein, the term “fixed dose” (also known as a “flat dose” or a “flat-fixed dose”) is used refer to a measured dose that is administered to an adult patient without regard for the weight or body surface area (BSA) of the patient. The fixed dose is therefore not provided as a mg/kg (weight-based) dose, or as a mg/m² (BSA) dose, but rather as an absolute amount of an agent (e.g., mgs of the anti-ErbB3 antibody) to be administered to an adult patient in a single administration.

### IV. Outcomes

A patient treated in accordance with the disclosed protocols may exhibit CR, PR, or SD with respect to target lesions. In another embodiment, the patient so treated experiences tumor shrinkage and/or decrease in growth rate, i.e., suppression of tumor growth. In another embodiment, tumor cell proliferation is reduced or inhibited. Alternately, one or more of the following can indicate a beneficial response to treatment: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent. Other indications of a favorable response include reduction in the quantity and/or size of measurable tumor lesions or of non-target lesions.

### V. Kits and Unit Dosage Forms

Also provided are kits that include, in an inner container (e.g., a vial) contained within an outer container (e.g., a bag, clamshell or box), a composition comprising an anti-ErbB3 antibody comprising CDRH1, CDRH2, and CDRH3 sequences comprising the amino acid sequences set forth in SEQ ID NO:1 (CDRH1) SEQ ID NO:2 (CDRH2) and SEQ ID NO:3 (CDRH3), and CDRL1, CDRL2, and CDRL3 sequences comprising the amino acid sequences set forth in SEQ ID NO:4 (CDRL1) SEQ ID NO:5 (CDRL2) and SEQ ID NO:6 (CDRL3) and a pharmaceutically acceptable carrier, in a therapeutically effective unit dosage form (e.g., as a single dose) for use in the preceding methods. Optionally, the anti-ErbB3 antibody is seribantumab. Unit dosage forms will typically comprise an amount of drug, optionally slightly above the dosage amount (e.g., 3000 mg) to facilitate removal of the required amount from the inner container. This dosage amount may comprise multiple vials, e.g., 12×10.1 mL vials or 6×20 mL vials. Each vial in a kit should comprise the same lot number. The kits can optionally also include instructions, comprising, e.g., administration parameters and schedules, to allow a practitioner (e.g., a physician or nurse) to administer the antibody composition (and other drugs, if any) contained therein to NSCLC patients in accordance with the methods taught herein. In one embodiment, the kit further comprises docetaxel and/or pemetrexed, e.g., each in a separate container, optionally in single dose unit dosage form. The kit may further contain diluents, instruments, or devices necessary for administering the pharmaceutical composition(s) e.g., one or more of a container of sterile diluent, e.g., saline or dextrose solution for injection; a syringe or syringes (e.g. pre-filled syringes); a catheter, a hypodermic (IV) needle, an IV infusion set.

The following examples are merely illustrative and should not be construed as limiting the scope of this disclosure in any way as many variations and equivalents will become apparent to those skilled in the art upon reading the present disclosure.

All patents, patent applications and publications cited herein are incorporated herein by reference in their entireties.

### EXAMPLES

### Methods

Heregulin (HRG) RNA-ISH is performed as described below and in pending international application No. PCT/US2014/072594, “Biomarker Profiles for Predicting Outcomes of Cancer Therapy with ErbB3 Inhibitors and/or Chemotherapies,” filed 29 Dec. 2014, with the exception of the core needle biopsy analysis in Example 3.

RNA-ISH Assay

In this assay, FFPE tumor samples are scored for HRG RNA levels using the following variant of an Advanced Cell Diagnostics® (“ACD” Hayward, Calif.) RNAscope® assay. Specifically, cells are permeabilized and incubated with a set of oligonucleotide “Z” probes (see, e.g., U.S. Pat. No. 7,709,198) specific for HRG. Using “Z” probes, as well as using multiple sets of probes per transcript, increases the specificity of the assay over standard ISH methods. One HRG probe set that can be used in this assay is ACD Part Number 311181. Another HRG probe set prepared by ACD (and used in RNAscope® assays) includes 62 probes (31 pairs), each 25 bases in length, that target a 1919 base long region of the HRG transcript comprising nucleotides 442-2977 of SEQ ID NO:42 and that together detect 15 separate HRG isoforms (α, β1, β1b, β1c, β1d, β2, β2b, β3, β3b, γ, γ2, γ3, ndf43, ndf34b, and GGF2). Following Z probe incubation, a pre-amplifier is added that can only hybridize to a pair of adjacent Z probes bound to the target transcript. This minimizes amplification of non-specific binding. Several sequential amplification steps are then performed based on sequence-specific hybridization to the pre-amplifier, followed by enzyme-mediated chromogenic detection that enables semi-quantitative measurement of HRG RNA levels in the tumor tissue.

Step 1: FFPE tissue sections are deparaffinized and pretreated to block endogenous phosphatases and peroxidases and to unmask RNA binding sites. Step 2: Target-specific double Z probes are applied, which specifically hybridize to the target RNA at adjacent sequences. Step 3: Targets are detected by sequential applications of a preamplifier oligonucleotide, amplifier oligonucleotides, a final HRP-conjugated oligonucleotide, and DAB. Step 4: Slides are visualized using a light microscope and scored by a pathologist.

To score the assay, a reference tissue microarray (TMA) of four cell lines is stained alongside the tumor sample. These cell lines express different levels of HRG, ranging from low to high. A pathologist then assigns the patient sample a score based on a visual comparison with the reference TMA.

1. Sample Preparation and Staining

Patient sample preparation and pathologist review procedures are similar to qIHC assays. Upon biopsy or surgical resection, patient tumor samples are immediately placed in fixative (10% neutral buffered formalin) typically for 20-24 hours at room temperature. Samples are then transferred to 70% ethanol and embedded in paraffin as per standard hospital procedures. Before the assay is performed, 4μm sections of the sample are prepared and mounted on positively charged 75×25 mm glass slides. These are baked for improved tissue adhesion (10-30 min at 65° C.), dipped in paraffin for tissue preservation, and stored at room temperature under nitrogen. One of the sections is used for routine H&E staining, which a pathologist reviews for tumor content, quality, and clinical diagnosis. The pathologist differentiates areas of tumor, stroma, and necrosis. Following this review, an adjacent or nearby tissue section (within 20 μm of the H&E section) is used for the assay.

Pretreat solutions, target probes, and wash buffers for RNAscope® assays are obtained from ACD. The assay can be run manually, or using a VENTANA autostainer (Discovery XT). For the manual assay, 40° C. incubations are performed in a metal slide tray inside a HybEZ oven (ACD). For the automated assay, incubation temperatures are controlled by the autostainer. ACD software is usede to run the RNAscope® assays on the VENTANA autostainer.

To begin the assay, samples are deparaffinized by baking at 65° C. for 30 min, followed by sequential immersion in xylenes (2×20 min) and 100% ethanol (2×3 min). After air-drying, tissues are covered with Pretreat1 solution, which blocks endogenous enzymes (phosphatases and peroxidases which would produce background with chromogenic detection reagents), incubated for 10 min at room temperature, then rinsed twice by immersion in dH₂O. Slides are then incubated in boiling Pretreat2 solution for 15 min, which unmasks binding sites, and transferred immediately to containers of dH₂O.

After washing by immersion in dH₂O (2×2 min), tissue is covered with Pretreat3 solution and incubated in a HybEZ oven at 40° C. for 30 min. Pretreat3 solution contains a protease, which strips the RNA transcripts of protein and exposes them to the target probes. After washing the slides 2×2 min in dH₂O, the tissues are covered with the 15 isoform-detecting HRG RNAscope® probes described above. Serial tissue sections are incubated with positive control probes (protein phosphatase 1B (PP1B) ACD Part Number 313901), negative control probes (bacterial gene DapB—ACD Part Number 310043), or HRG probes for 2 h at 40° C. Slides are washed (2×2 min) with 1× RNAscope® wash buffer before incubating with Amp1 reagent. Amp1 incubation conditions (30 min, 40° C.) favor binding only to pairs of adjacent probes bound to RNA transcripts. Slides are washed by immersion in RNAscope® wash buffer before incubating with subsequent amplification reagents.

For signal amplification, each of the sequentially applied reagents binds to the preceding reagent and amplifies the signal present at the previous step. Amplification steps may include Amp2 (15 min, 40° C.), Amp3 (30 min, 40° C.), Amp4 (15 min, 40° C.), Amp5 (30 min, room temperature), and Amp6 (15 min, room temperature). The final reagent, Amp6, can be conjugated to horseradish peroxidase (HRP). To visualize the transcripts, the slides are then incubated with the ACD staining reagent, which contains diaminobenzidine (DAB), for 10 min at room temperature. Chromogen development is stopped by rinsing with dH₂O. Nuclei are then counterstained with hematoxylin, which is blued with dilute ammonium chloride. Stained slides are immersed in 80% ethanol (2×5 min), 100% ethanol (2×5 min), and xylenes (2×5 min) before coverslipping with Cytoseal non-aqueous mounting medium (Thermo Scientific, 8312-4).

2. Generation of Biomarker Values

The biomarker values to be generated are a composite of pathologist scores. To score the assay, a TMA comprising plugs of four different cell lines is included in each staining run. Cell line plugs are prepared prior to generating a TMA. Cultured cells grown to a sub-confluent density are harvested by trypsinization, rinsed in PBS, and fixed for 16-24 hr at 4° C. before rinsing in PBS and resuspending in 70% ethanol. Cells are then centrifuged for 1-2 minutes at approximately 12,000 rpm to produce a dense cell pellet, which is then coated with low-melting point agarose. The agarose pellets are stored in 70% ethanol at 4° C., and embedded in paraffin before constructing the TMA.

The arrays are constructed, e.g., using a Manual Tissue Arrayer (MTA-1, Beecher Instruments), with which a 0.6 mm punch is used to take a portion of the cell pellet and plug it into an empty recipient paraffin block. The pathologist uses the images of the TMA to provide a score ranging from 0 (undetectable) to 4 (high). The pathologist provides two scores for the top two populations of tumor cells, and one score for the top population of stromal cells (when available), along with the percentage of cells in each population. So, for example, a patient sample may have 20% tumor with a score of 3, 40% tumor with a score of 2, and 60% stroma with a score of 2. Scores are provided for the target probe (HRG), as well as the positive control probe (PP1B) and the negative control probe (DapB).

### Example 1

### Seribantumab Shows In Vitro and In Vivo Single Agent Activity Against Growth of Lung Cancer Cell Lines that are Responsive to Heregulin (HRG)

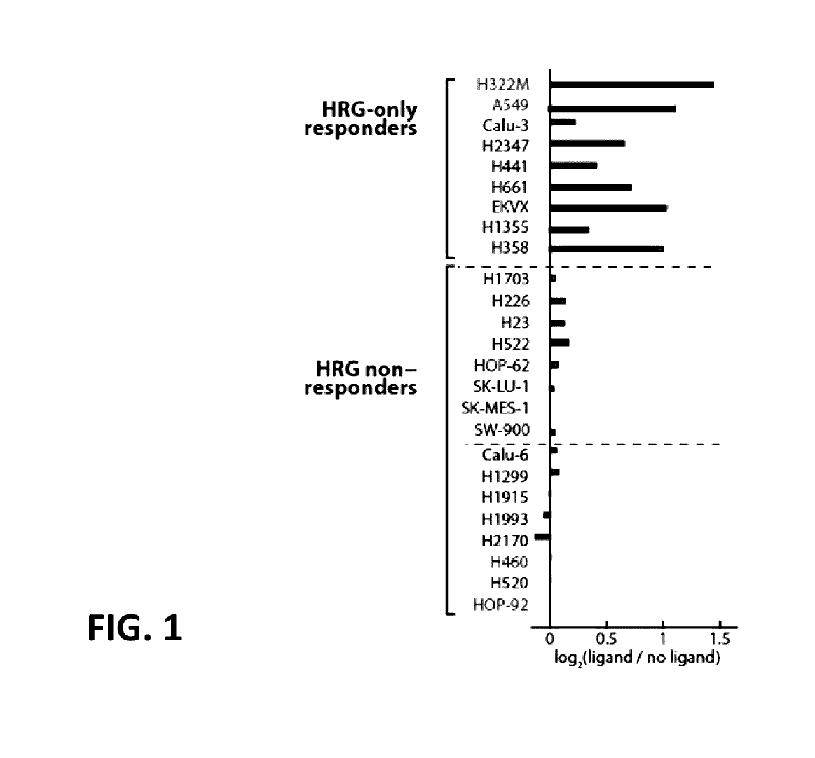

RNA-ISH assays and biomarker analysis are performed as described above. These studies indicate that 9 out of 25 EGFR wild-type NSCLC cell lines are responsive to HRG: they exhibit increased cell proliferation in response to exogenously added HRG, as measured by a CellTiter Glo® luminescent cell viability assay (Promega) using 3D spheroid cultures (FIG. 1).

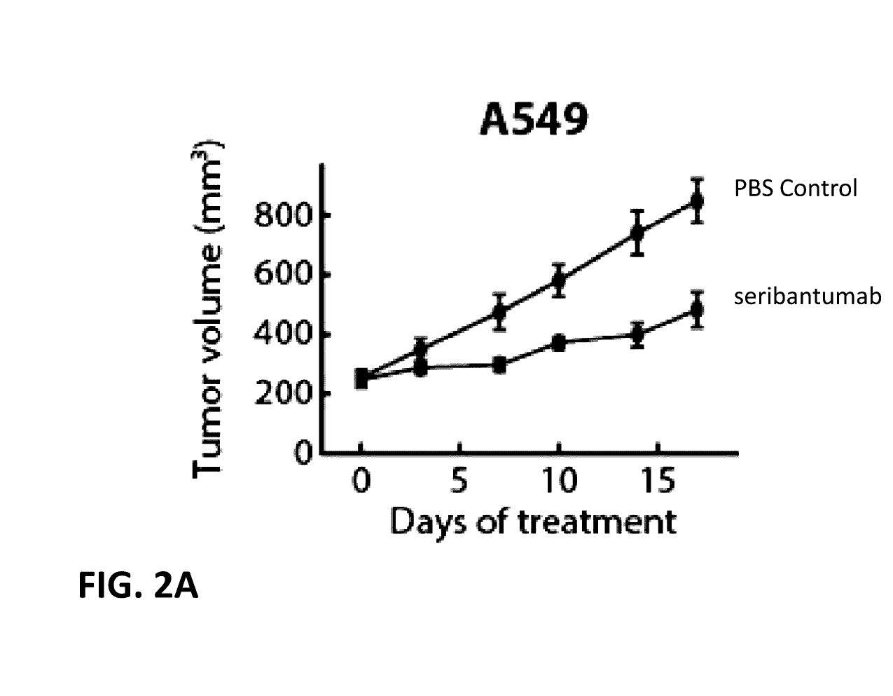

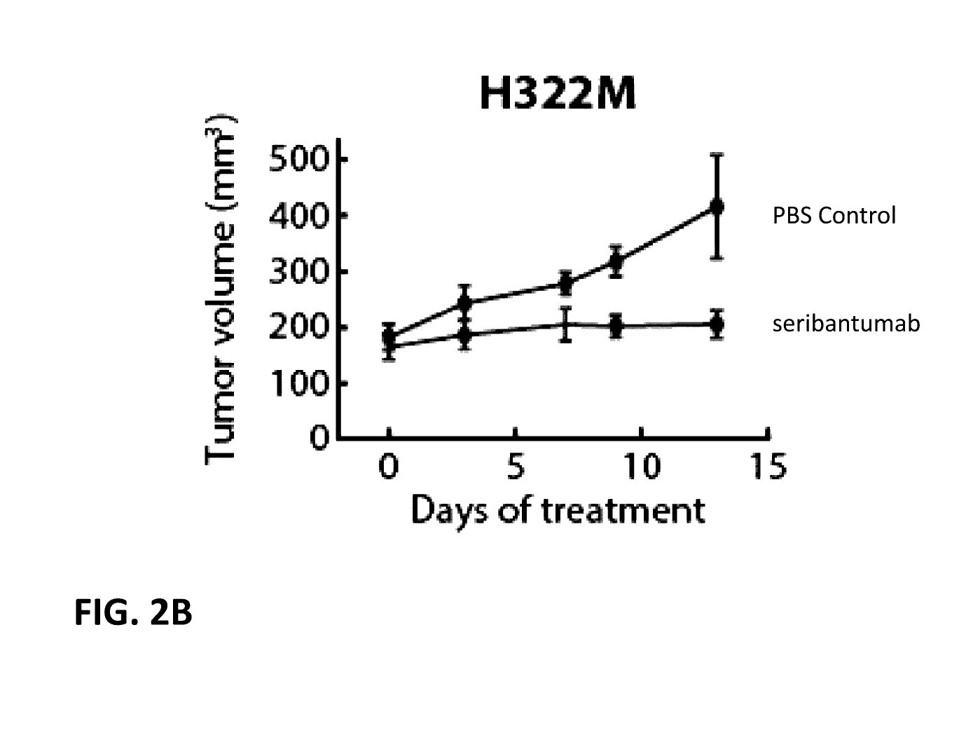

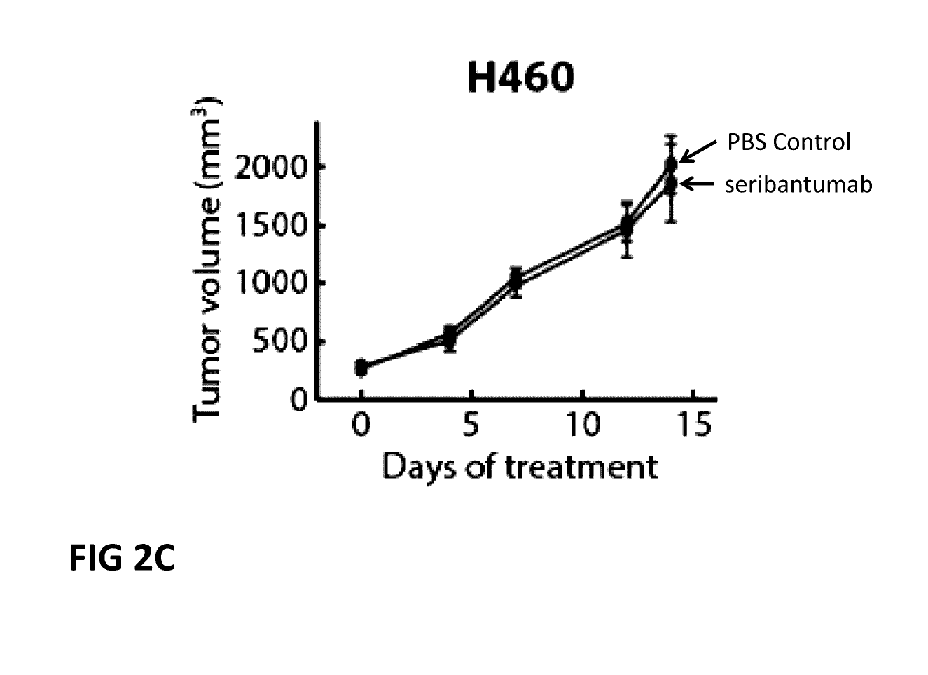

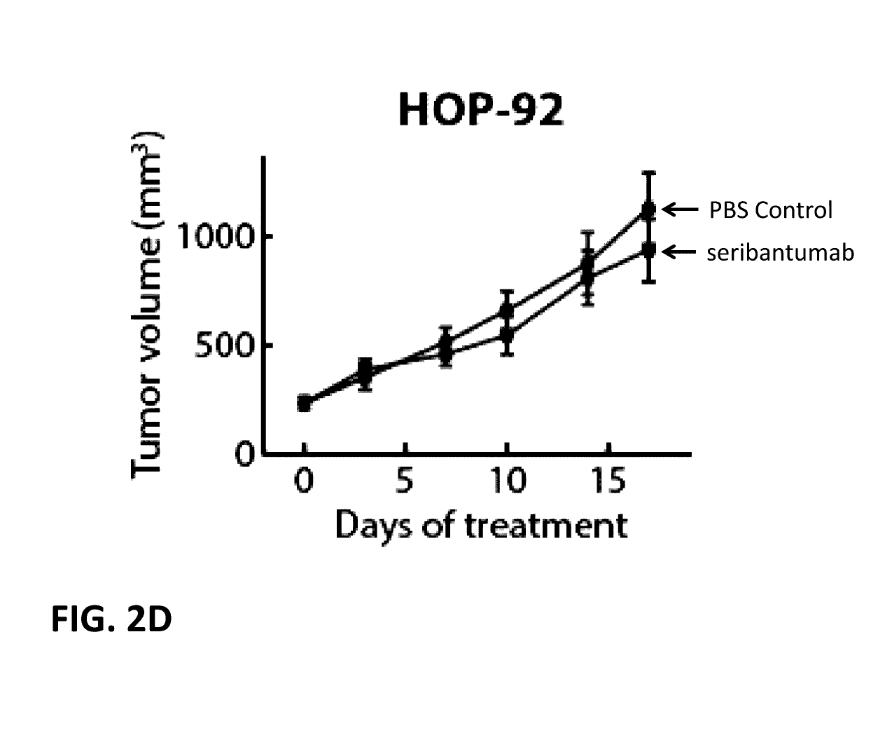

Two HRG-responsive cell lines and two non-responsive cell lines were selected to assess the single agent activity of seribantumab in subcutaneous mouse xenografts. The mice were dosed with 300 μg seribantumab every three days (Q3D). As shown in FIGS. 2A and 2B, the HRG-responsive cell lines(A549 and H322M, respectively) responded to seribantumab as a single agent in vivo. In contrast, H460 and Hop92, which were not responsive to HRG in vitro, did not respond to seribantumab in vivo (FIGS. 2C and 2D, respectively). High tissue HRG mRNA levels were measured in the seribantumab-responsive xenograft tumors. Interestingly, both human HRG mRNA, indicative of autocrine HRG signaling, and mouse HRG mRNA, indicative of stroma-derived paracrine signaling, were observed in the HRG-responsive tumors. These data indicate that a subset of EGFR wild-type NSCLC cell lines are responsive to HRG, that these cell lines elicit the production of HRG, and that the presence of HRG in tissue appears to be necessary for seribantumab response in vivo, further supporting exclusion of patients whose tumors do not express HRG.

### Example 2

### Seribantumab Treatment can Overcome HRG-Induced Resistance to Pemetrexed and Docetaxel in Lung Cancer Cell Lines

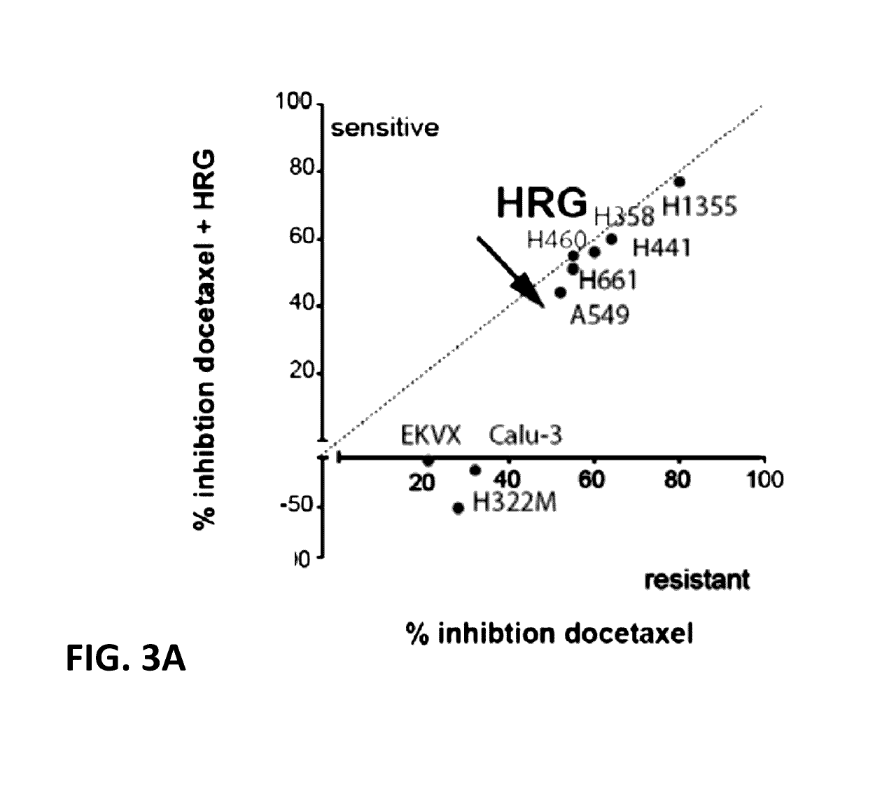

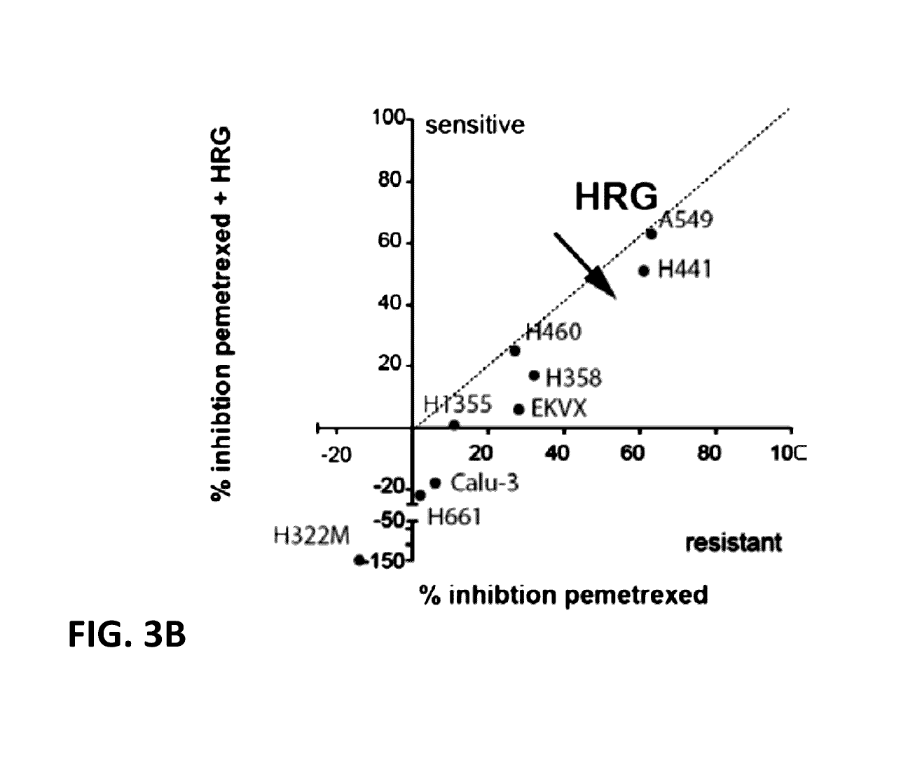

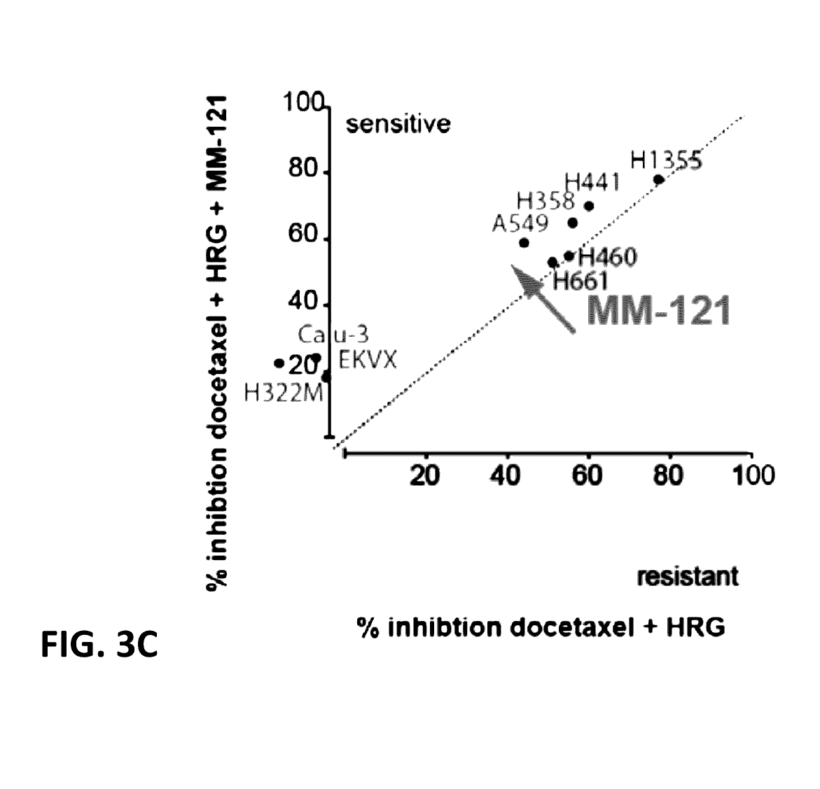

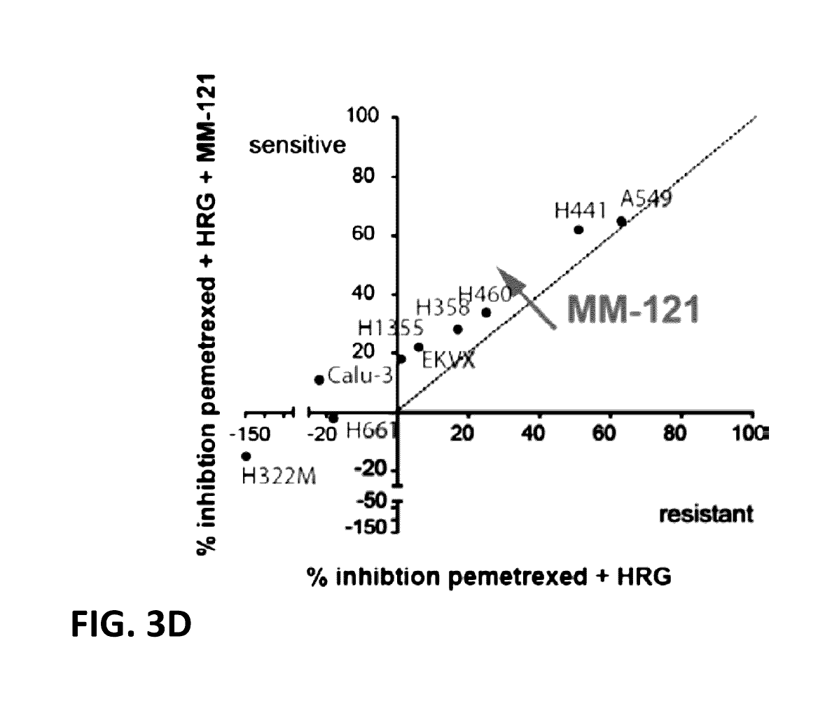

As depicted in FIG. 3A-3D, HRG induces resistance to pemetrexed and docetaxel in a panel of 9 lung cancer cell lines. HRG-driven ErbB3 signaling mediates survival signaling through the PI3K/AKT pathway and has been implicated as a general mechanism that imparts insensitivity to cytotoxic chemotherapy. As shown in FIGS. 3A and 3B, HRG induces resistance to pemetrexed and docetaxel in a subset of EGFR wild-type NSCLC cell lines. Proliferation was measured, in the presence or absence of HRG, in a panel of nine cell lines using 3D spheroid cultures. Full dose response curves were obtained but results are only shown for a single relevant dose of chemotherapy. In three of these cell lines—those most responsive to HRG—inhibition of cell viability by both docetaxel and pemetrexed was decreased upon the addition of HRG. In fact, HRG induced proliferation even in the presence of chemotherapy, as noted by the negative values for % inhibition. Importantly, when seribantumab was added in addition to HRG, sensitivity to both docetaxel and pemetrexed was restored in these cell lines (FIGS. 3C and 3D).

### Example 3

### HRG mRNA Expression Levels in NSCLC Tissue Samples

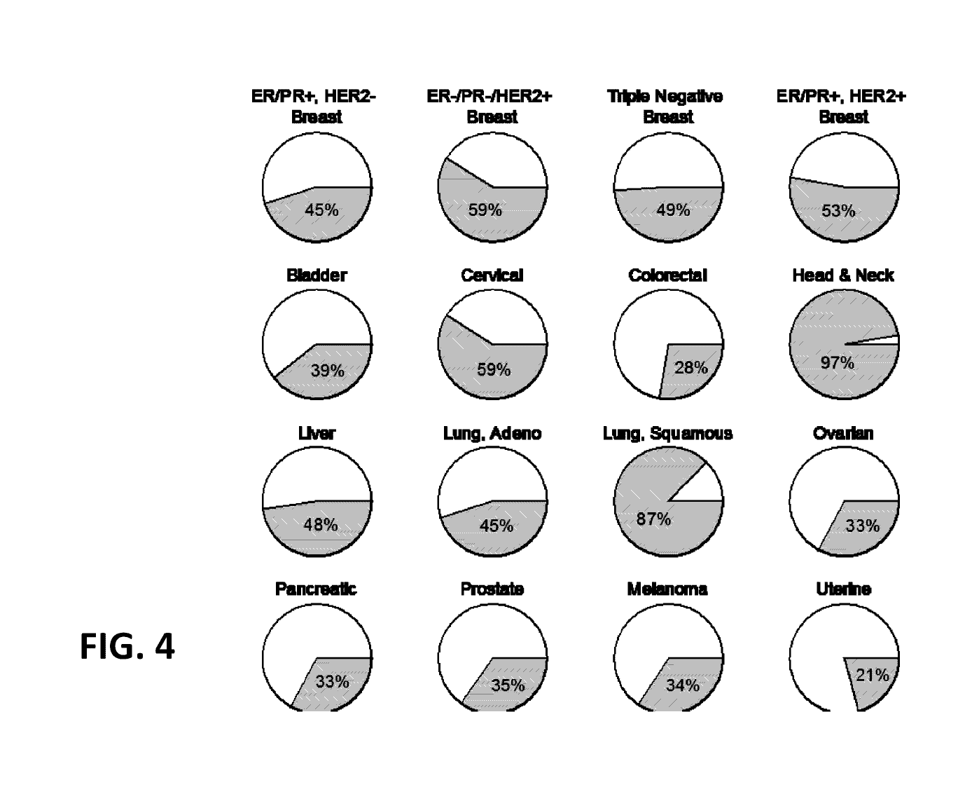

Analysis of tumor samples from previous randomized phase II clinical trials of seribantumab in breast and ovarian cancer indicated that a CT level of HRG expression of −5 relative to reference genes as measured by quantitative RT-PCR (per PCT/US2014/072594, discussed above) was a threshold value for seribantumab activity. In patients with HRG expression at or above the threshold (≧−5), increased PFS was observed in patients treated with seribantumab co-administered with standard-of-care therapy. Since this threshold roughly corresponds to the presence of detectable HRG-encoding RNA, The Cancer Genome Atlas (TCGA; http://cancergenome.nih.gov/) dataset was analyzed to determine the prevalence of detectable HRG expression in a wide variety of solid tumors (FIG. 4). The data suggest that NSCLC is an indication in which HRG-driven ErbB3 signaling is particularly prevalent.

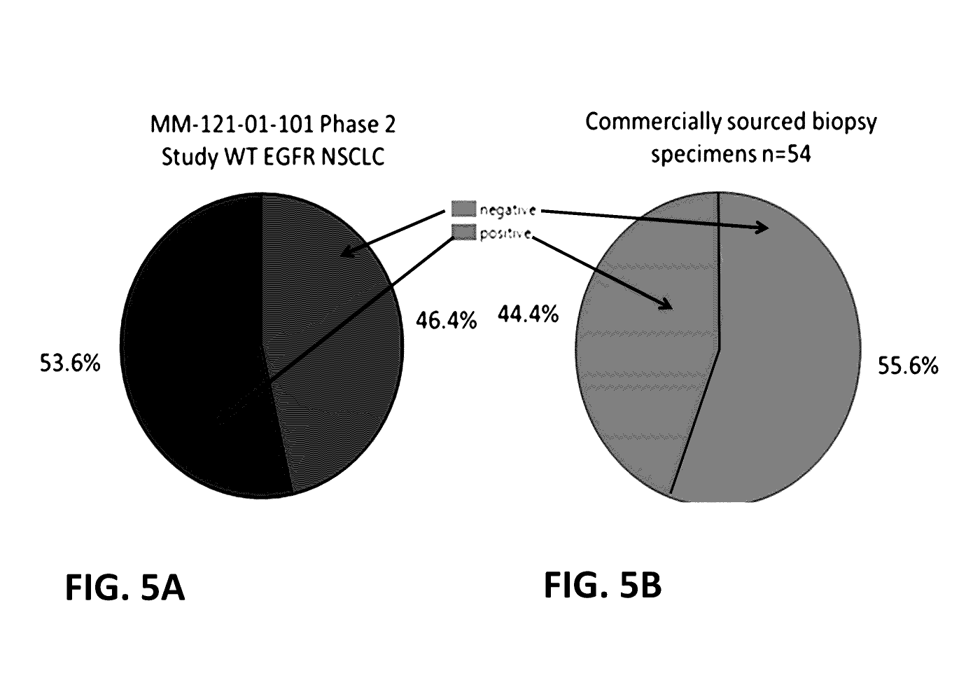

In addition, HRG expression was assessed using an RNA in situ hybridization (RNA-ISH) assay (also per PCT/US2014/072594) in pre-treatment core needle biopsies obtained from patients enrolled in a study of seribantumab in EGFR wild-type NSCLC (MM-121-01-101). Overall, 54% of the samples scored 1+ (i.e., 1-3 dots/cell (visible at 20-40× magnification) or higher (FIG. 5A). Furthermore, the analysis was expanded and an additional 53 archival lesions and biopsies were analyzed that were procured from Cureline, Inc. (San Francisco, Calif.) (FIG. 5B). Comparable to the findings in the MM-121-01-101 lung study, the prevalence of HRG mRNA by RNA-ISH with a score of >1+ was found to be between 44-54%, and correlated with increased PFS from the addition of seribantumab.

### Example 4

### Determination of a Seribantumab Dose for Combination with Docetaxel or Pemetrexed

Population pharmacokinetic (PK) analyses support using a fixed dosing regimen for seribantumab.

Analysis by simulation: To evaluate optimal dosing regimens, population analysis was used to estimate the point estimates and variabilities of pharmacokinetic parameters, and to evaluate the source of the variabilities, including their relationships with body weight. The resulting estimates were used to compare fixed dosing and weight-based dosing regimens. For fixed dosing strategies, comparable dose is simulated by assuming the weight-based dose times the median of weight in the population (72 kg), rounded to the next 500 mg (vial size). The simulation results show comparable variability between both fixed-dosing and weight-based dosing regimens, suggesting no benefits of reduced PK variability with weight-based dosing (higher concentrations are predicted for the dose regimens of 10 mg/kg equivalent only because of rounding up doses to the next 500 mg). For example, a weight-based dosing of 20 mg/kg Q2W and a corresponding fixed dose of 1.5 g Q2W have comparable maximum, minimum, and average steady-state concentration levels and variability. This result can be explained as a consequence that clearance increased less than proportionally to weight (i.e., the estimated proportionality between log₁₀ of clearance and weight was 0.203). This proportionality results in higher-weight patients being overdosed by a weight-based regimen (which assumed a proportionality constant of one between log₁₀ of clearance and weight).

A simulation study, conducted by comparing the simulated pharmacokinetics (averaged and minimum concentration) at different dose intervals, indicates an every 3 week regimen is optimal. A dose regimen of 3 g Q3W is predicted to have: 1) comparable maximum concentration (Cmax) to 40 mg/kg Q3W; 2) comparable minimum concentration (Cmin) to 20 mg/kg Q2W; and 3) average steady-state concentration in between 20 mg/kg Q2W (the dose studied in previous NSCLC study) and 20 mg/kg Q1W (the dose studied in previous ovarian and breast cancer studies). Therefore, this simulation study suggests that a seribantumab dose regimen of 3 g Q3W should improve compliance and convenience while maintaining the pharmacokinetic levels within the bounds of the exposures observed from previously studied effective seribantumab doses (40 mg/kg loading+20 mg/kg Q1W or +20 mg/kg Q2W). To evaluate the contribution of loading dose, concentration trajectories of simulated dose regimens with and without loading dose are compared. The loading dose is limited to a maximum of 3 g (a corresponding fixed dose for a 40 mg/kg). The results show comparable pharmacokinetics with and without a loading dose, and therefore, support the regimen without loading dose.

Experimental: The pharmacokinetics of seribantumab were evaluated using population pharmacokinetic analysis from 499 patients who had been treated with seribantumab. 4925 data points from the combined phase I and phase II studies of seribantumab were analyzed. These pharmacokinetic data were described using a two-compartment model, with estimated parameters provided in Table 1. Covariate selection evaluated potential relationships between baseline covariates (sex, race, age, weight, intended-dose, and study/indication) and volume of distribution and clearance. The results indicated significant relationships between weight, sex, and clearance, with the final parameter estimates provided in Table 1. The model assumed a proportional relationship between the log of clearance (CL) and weight, and obtained an estimated proportionality constant of 0.203. In the presence of the relationship between weight and clearance, no significant relationship between volume (V) and weight (WT) were observed.

TABLE 1
Final parameter estimates from population PK analysis of seribantumab

| | (Estimated) |
|---|---|
| Parameters | values |
| Number of | 499 |
| patients | |

Fixed effects

| | |
|---|---|
| CL (L/wk) | 3.15 |
| V (L) | 3.23 |
| Q (L/wk) | 2.92 |
| V2 (L) | 2.68 |

Random effects

| | |
|---|---|
| Omega CL (%) | 36% |
| Cov CL and V (%) | 27% |
| Omega V (%) | 37% |

Sigma

| | |
|---|---|
| Additive | 25.18 |
| Proportional | 0.23 |

Covariate selection

| | |
|---|---|
| WT-CL | 0.203 |
| SEX-CL | 0.255 |
| WT-V | 0.002 |

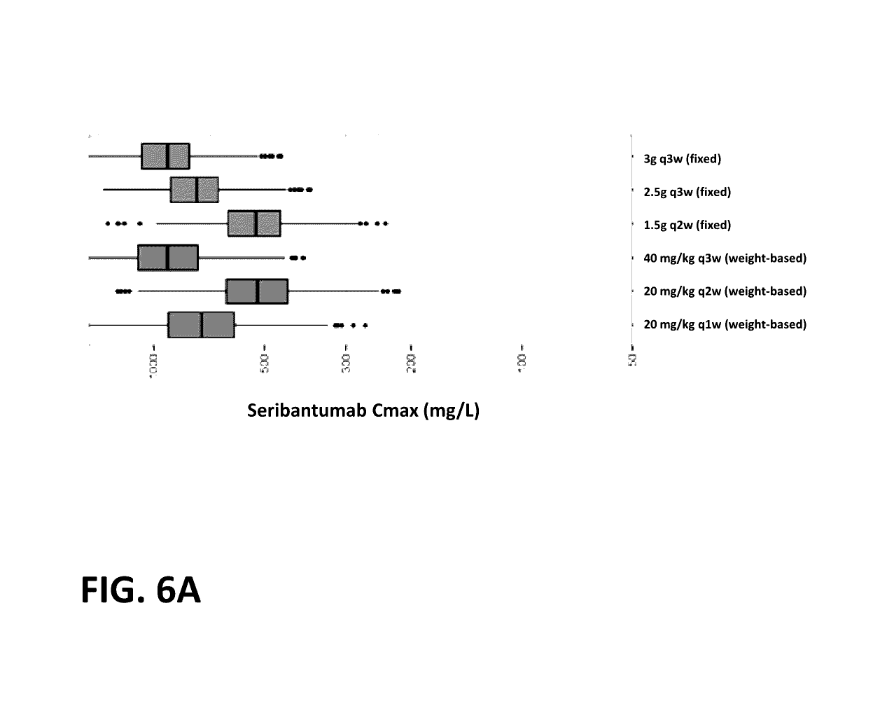

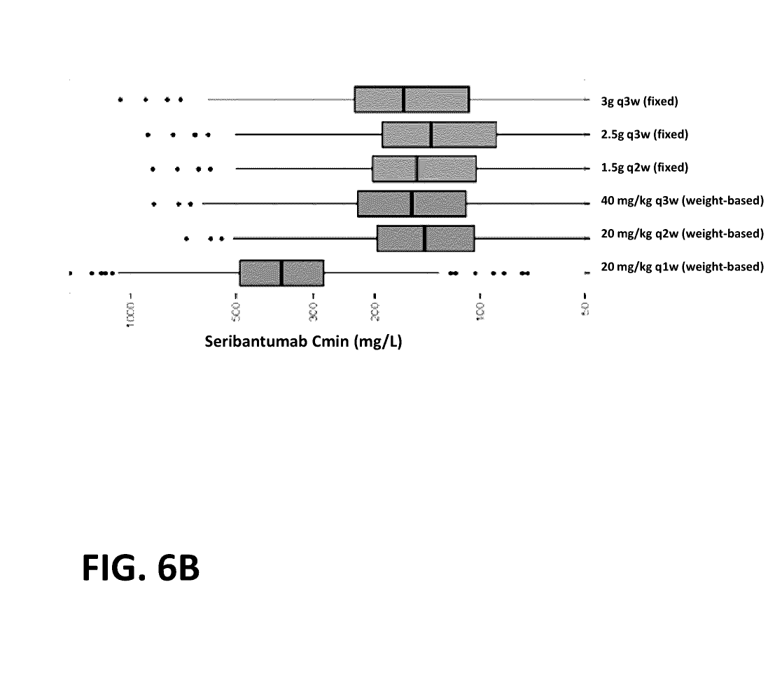

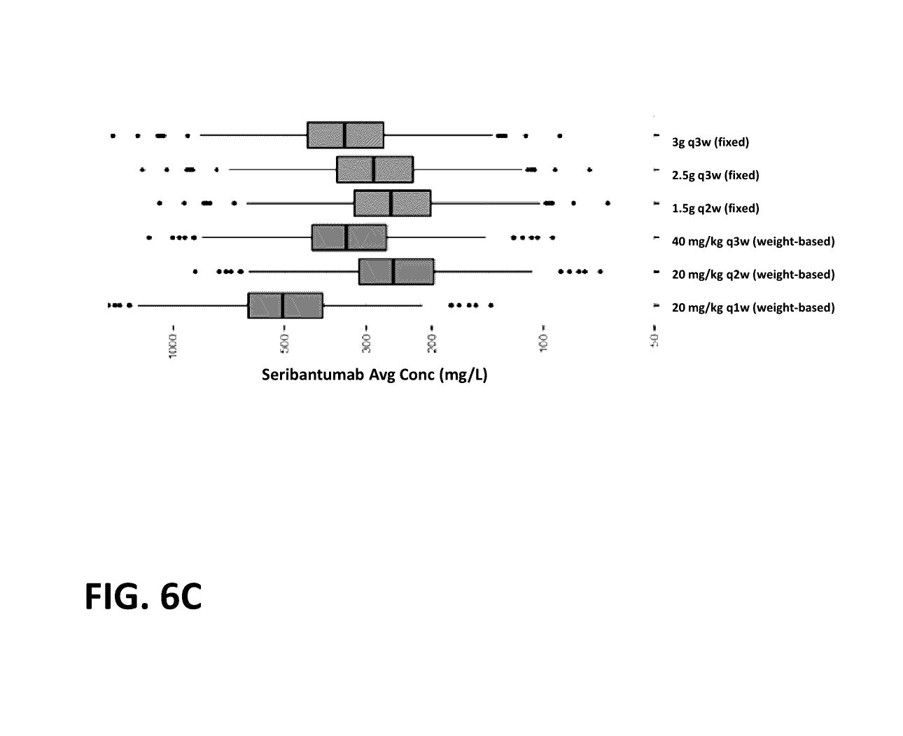

To evaluate the benefit of weight-based dosing, a simulation study was conducted by comparing pharmacokinetics with weight-based and fixed-dose regimens. Post-hoc estimates of PK parameters from each of the 499 patients were used in the simulation. The simulated dose for the fixed dosing regimen was chosen by rounding up to the closest 500 mg dose unit. The simulation results showed comparable variability between both fixed-dosing and weight-based dosing regimens, suggesting no benefits of the reduced PK variability with weight-based dosing (FIGS. 6A-6C). For example, a weight-based dosing of 20 mg/kg Q2W and a corresponding fixed dose of 1.5 g Q2W have comparable maximum, minimum, and average steady-state concentration levels and variability. The result can be explained in that estimated proportionality between log of CL and weight is 0.203, and therefore, a weight-based regimen (which assumed a proportionally constant of one between log of CL and weight) would tend to overdose higher-weight patients. To evaluate the optimization of seribantumab dosing regimens for improved compliance and simplicity, a simulation study was conducted by comparing the simulation pharmacokinetics (averaged and minimum concentration) by different dose intervals. The results showed the potential to optimize the dosing frequency to once every 3 weeks. A dose regimen of 3000 mg Q3W is predicted to have: 1) a comparable maximum concentration (Cmax) to 40 mg/kg Q3W, a dose level previously used as a loading dose for weight-based and weekly seribantumab dosing regimens; 2) a comparable minimum concentration (Cmin) to 20 mg/kg Q2W which was the dose used in the previous seribantumab study in NSCLC in combination with 100 mg erlotinib; and 3) an average steady-state concentration that is in between 20 mg/kg Q2W and 20 mg/kg Q1W which is the previously studied regular dose for seribantumab following the 40 mg/kg loading dose in combination with chemotherapy. Therefore, this simulation study suggests that a seribantumab dose regimen of 3000 mg Q3W has a potential to improve compliance while maintaining the pharmacokinetic levels within the bounds of the exposures observed from previously studied seribantumab doses (40 mg/kg+20 mg/kg Q1W and 20 mg/kg Q2W). In addition, no MTD was identified when seribantumab was co-administered with standard doses of pemetrexed, paclitaxel or cabazitaxel. In these studies, seribantumab was co-administered with full doses of the chemotherapy agents (pemetrexed, paclitaxel or cabazitaxel) at 40 mg/kg as a loading dose followed by weekly doses of 20 mg/kg. The loading dose of 40 mg/kg equals 3000 mg in an average patient weighing 75 kg. As such, the cumulative seribantumab dose proposed for this study, 3000 mg seribantumab Q3W as a fixed dose, does not exceed previously tested dose regimens for seribantumab in combination with pemetrexed.

Accordingly, seribantumab will be administered at a fixed dose of 3 g/3000 mg on day 1 of each 21-day cycle in sync with the chemotherapy regimens outlined in the study below.

### Example 5

### Study Design for Treatment of NSCLC

Title: A Phase 2 Study of Seribantumab (MM-121) in Combination with Docetaxel (D) or Pemetrexed (P) versus D or P Alone in Patients with Heregulin Positive (HRG+), Locally Advanced or Metastatic Non-Small Cell Lung Cancer.

BACKGROUND: The role of the HER3 receptor and its ligand heregulin (HRG) in the progression of multiple cancers has been well established. Seribantumab (MM-121) is a fully human, monoclonal IgG2 antibody that binds to the HRG domain of HER3, blocking HER3 activity. In retrospective analyses of prior seribantumab Phase 2 studies, high levels of HRG mRNA appeared to predict poor outcome when patients received standard of care (SOC) treatment. Addition of seribantumab to SOC improved progression-free survival (PFS) in patients with HRG positive (HRG+) tumors, consistent with the hypothesis that blockade of HRG-induced HER3 signaling by seribantumab can restore sensitivity to SOC impacted by HRG.

METHODS: In the current randomized, open-label, international, Phase 2 study, NSCLC patients will be screened for HRG using an RNA in situ hybridization assay on a recent biopsy tissue sample. Approximately 560 patients will be screened to support randomization of approximately 280 HRG+ patients in a 2:1 ratio to receive seribantumab plus investigator's choice of docetaxel (D) or pemetrexed (P), or D or P alone. Patients will be wild-type for EGFR and ALK and will have progressed following one to three systemic therapies for locally advanced and/or metastatic disease, including one platinum-containing regimen and anti-PD-1/PD-L1 where available and clinically indicated. The primary endpoint is overall survival (OS). Secondary endpoints include PFS, objective response rate and time to progression.

Approximately 227 OS events are required to have ≧80% power to detect a 3-month improvement in median OS with seribantumab plus D or P versus D or P alone with a baseline median OS assumption of 6 months (hazard ratio ≦0.67), using a one-sided, stratified log-rank test at a significance level of 0.025. An interim analysis for stopping due to futility or efficacy will be conducted when 50% of final OS events have been reported.

This study is a randomized, open-label, international, multi-center, phase II study in adult patients with NSCLC that has progressed following no more than two systemic therapies for locally advanced or metastatic disease, of which one must have been a platinum-based doublet therapy.

Following signing informed consent and evaluation of initial eligibility criteria, all patients will provide a tissue sample (which meets the requirements for collection and processing as outlined in the study lab manual) to a central lab facility for HRG testing. It is important that no systemic therapy is administered between the date of acquisition of the tissue sample and screening for this study in order to accurately assess a patient's HRG status. If adequate tissue is not available, patients should undergo a fine needle aspirate (FNA) or core needle biopsy (CNB) to acquire the necessary tissue for HRG testing. For these procedures, investigators are asked to choose an easily accessible tumor lesion to minimize any possible risk associated with the collection of the tissue. As a general guideline, if the selected procedural location of the core needle biopsy or FNA has an established serious complication rate of >2% at the institution completing the procedure, this is considered a high risk procedures and should be avoided. Upon receipt of a tissue sample at the central lab, the investigational site will be informed of the results within 7 days. Patients with a positive HRG status will be eligible for the interventional study population. Patients with tumors that show no staining for HRG will not continue further screening procedures and will be eligible for the observational group as outlined below.

### Observational Group

Baseline data will be collected which includes demographics, disease characteristics and previous treatments. In addition, data regarding subsequent anti-cancer therapies received and OS will be collected. Patients are free to participate in any study and seek any care suitable.

### Interventional Group

By the time all screening procedures have been completed and determination of eligibility for treatment randomization (HRG positive, interventional group), the investigator must select the chemotherapy backbone (docetaxel or pemetrexed) most appropriate for each patient based on current presentation and medical history. Patients will be randomized in a 2:1 ratio (experimental arm versus comparator arm) using an Interactive Web Response System (IWRS). Randomization will be stratified based on the chemotherapy backbone (docetaxel or pemetrexed) and number of prior systemic therapies for locally advanced or metastatic disease (1 or 2). Within the interventional group, patients will be assigned to Arm A or Arm B:

### Interventional Arm A (Experimental Arm):

Seribantumab: fixed dose of 3000 mg (12×10.1 mL vials; 6×20 mL vials) intravenously (IV) on day 1 of each 21-day cycle
Docetaxel: 75 mg/m² IV on day 1 of each 21-day cycle
OR
Seribantumab: fixed dose of 3000 mg (12×10.1 mL vials; 6×20 mL vials) IV on day 1 of each 21-day cycle
Pemetrexed: 500 mg/m² IV on day 1 of each 21-day cycle

### Interventional Arm B (Comparator Arm):

Docetaxel: 75 mg/m² IV on day 1 of each 21-day cycle
OR
Pemetrexed: 500 mg/m² IV on day 1 of each 21-day cycle

Treatment must start within 7 days following randomization. Patients are expected to be treated until investigator-assessed progressive disease or unacceptable toxicity. Tumor assessments will be measured and recorded by the local radiologist every 6 weeks (+/−1 week) and evaluated using the RECIST guidelines (version 1.1). All patients, including any patient that comes off treatment for reasons other than RECIST 1.1 assessed progressive disease, should have an additional scan 6 weeks (+/−1 week) following treatment termination. In addition, an independent central review of scans will be conducted to support secondary efficacy objectives. All images for patients in the interventional group will be submitted to a central imaging facility for this purpose and will be assessed by independent reviewers in accordance with the Imaging Charter. After patients come off treatment, survival information and information about subsequent therapies will be collected until death or study closure, whichever occurs first.

Safety has been established for the combination of seribantumab+pemetrexed, and seribantumab has been administered in combination with taxanes (paclitaxel and cabazitaxel) at the standard doses with no maximum tolerated dose (MTD) reached. However, as no data is available for the combination of seribantumab and docetaxel, enrollment into this backbone will be paused after the twelfth patient has been randomized to docetaxel or seribantumab+docetaxel and completed one full cycle of treatment, and the emerging safety data on both arms will be reviewed by investigators, medical monitors and representatives from the sponsor. Additional input may be gathered from the DMC before continuing enrollment. The DMC will continue to monitor safety data in accordance with the DMC Charter on a quarterly basis.

### Inclusion Criteria

For inclusion in the trial, all patients will have/be: cytologically or histologically confirmed NSCLC, with either metastatic disease (stage IV); Stage IIIB disease not amenable to surgery with curative intent; disease progression or evidence of recurrent disease documented by radiographic assessment following the last systemic therapy; received one prior platinum-based regimen for the management of primary or recurrent disease; clinically eligible for intended chemotherapy, docetaxel or pemetrexed, once every three weeks per the investigator's judgment; available recent tumor specimen, collected following completion of most recent therapy; a lesion amenable to either core needle biopsy or fine needle aspiration; greater than or equal to eighteen years of age; and able to provide informed consent or have a legal representative able to do so. To be included in the interventional group, patients will have/be: a positive in situ hybridization (ISH) test for heregulin with a score of ≧1+, as determined by centralized testing; measureable disease in accordance with RECIST v1.1; ECOG performance status (PS) of 0 or 1; Screening ECG without clinically significant abnormalities; Adequate bone marrow reserve as evidenced by ANC >1,500/μl, platelet count >100,000/μl, and hemoglobin >9 g/dL; adequate renal function as evidenced by a serum/plasma creatinine <1.5×ULN for patients receiving docetaxel and a creatinine clearance ≧45 mL/min for patients receiving pemetrexed; for patients receiving pemetrexed: Aspartate aminotransferase (AST) and alanine aminotransferase (ALT) ≦2.5×ULN (≦5×ULN is acceptable if liver metastases are present); for patients receiving docetaxel: Aspartate aminotransferase (AST) and alanine aminotransferase (ALT) ≦1.5×ULN, Alkaline phosphatase (AP) <2.5 ULN and serum/plasma total bilirubin within normal institutional limits.

Women of childbearing potential, as well as fertile men and their partners, must be willing to abstain from sexual intercourse or to use an effective form of contraception during the study (an effective form of contraception is an oral contraceptive or a double barrier method) and for 90 days following the last dose of study drug(s), or greater, as in accordance with the label requirements or institutional guidelines for docetaxel/pemetrexed.

### Exclusion Criteria

Patients will meet all the inclusion criteria listed above and none of the following exclusion criteria:

a) Known Anaplastic Lymphoma Kinase (ALK) gene rearrangement or presence of exon 19 deletion or exon 21 (L858R) substitution of the EGFR gene

b) Pregnant or lactating

c) Prior radiation therapy to >25% of bone marrow-bearing areas

d) Received >2 prior systemic anti-cancer drug regimen for locally advanced disease
Maintenance therapy with pemetrexed following first-line treatment for Stage IIIB or Stage IV disease is counted as one line of therapy

e) Patients who have received prior docetaxel for advanced/metastatic disease are not eligible for the docetaxel-containing chemotherapy backbone

f) Patients who have received prior pemetrexed for advanced/metastatic disease and/or maintenance therapy are not eligible for the pemetrexed-containing chemotherapy backbone

g) Received other recent antitumor therapy including:
Investigational therapy administered within the 28 days or 5 half-lives, whichever is shorter, prior to the first scheduled day of dosing in this study
Radiation or other standard systemic therapy within 14 days prior to the first scheduled dose in this study, including, in addition (if necessary), the timeframe for resolution of any actual or anticipated toxicities from such radiation

h) CTCAE grade 3 or higher peripheral neuropathy

i) Presence of an unexplained fever >38.5° C. during screening visits that does not resolve prior
to the first day of dosing. If the fever and active infection have resolved prior to randomization,
the patient will be eligible. At the discretion of the investigator, patients with tumor fever may
be enrolled.

j) Symptomatic CNS metastases or CNS metastases requiring steroids

k) Use of strong CYP3A4 inhibitors for patients considered for the docetaxel backbone.

l) Any other active malignancy requiring systemic therapy

m) Known hypersensitivity to any of the components of MM-121 or previous hypersensitivity reactions to fully human monoclonal antibodies

n) History of severe allergic reactions to docetaxel or pemetrexed

o) Known hypersensitivity to polysorbate (Tween®) 80 or arginine

p) Clinically significant cardiac disease, including: symptomatic congestive heart failure, unstable angina, acute myocardial infarction within 1 year months of planned first dose, or unstable cardiac arrhythmia requiring therapy (including torsades de pointes).

q) Uncontrolled infection requiring IV antibiotics, antivirals, or antifungals, known human immunodeficiency virus (HIV) infection, or active B or C infection.

r) Patients who are not appropriate candidates for participation in this clinical study for any other reason as deemed by the investigator.

### Example 6

### Co-Administration of Seribantumab and Chemotherapeutics Abrogates HRG-Mediated Resistance to Said Chemotherapeutics in an Ovarian Cancer Mouse Xenograft Model

The anti-tumor efficacy of seribantumab and a chemotherapeutic agent (e.g. irinotecan, gemcitabine, or paclitaxel) either alone (i.e., as a monotherapy) or in combination, in tumor-bearing mice was evaluated using human ovarian epithelial carcinoma OVCAR8 cells (NCI) implanted as xenografts in nu/nu nude, Crl:NU-Foxn1^{nu} mice. In these xenograft studies, the mice were obtained from Charles River Laboratories. The mice were housed in Tecniplast® Individually Ventilated polycarbonate (Makrolon®) Cages (IVC) set in climate-controlled rooms and had free access to food and acidified water. A cell suspension of 8×10⁶ cells/mouse, mixed 1:1 in reduced growth factor Matrigel™ (BD Biosciences, Cat #354230) and PBS was implanted by subcutaneous injection into the left flank of female, 4-5 week old nu/nu nude, Crl:NU-Foxn1^{nu} mice. Tumors were allowed to reach 250 mm³ in size before randomization.

### Combination Therapy Study

A combination therapy study was performed to demonstrate the effects of various combinations of a fixed dose of seribantumab, irinotecan HCl, gemcitabine, and paclitaxel.

Mice were randomized as above into 8 groups of 10 mice each. Five groups were treated with i.p. doses of a single agent alone, as follows: (1) seribantumab (300 μg Q3D), (2) irinotecan HCl (6.25 mg/kg Q7D), (3) gemcitabine (25 mg/kg Q7D), (4) paclitaxel (10 mg/kg Q7D), or (5) PBS (Q3D) alone (Control). Three groups were treated with a combination therapy of (1) seribantumab and paclitaxel, (2) seribantumab and irinotecan HCl, and (3) seribantumab and gemcitabine, with the doses described above. Treatment continued for three weeks. Tumors were measured twice weekly and tumor volume calculated.

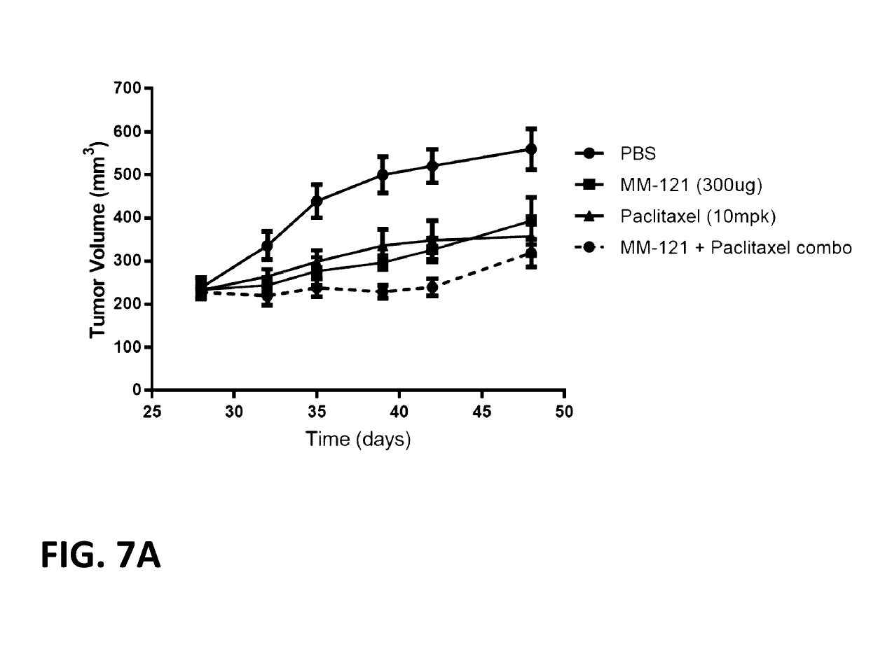

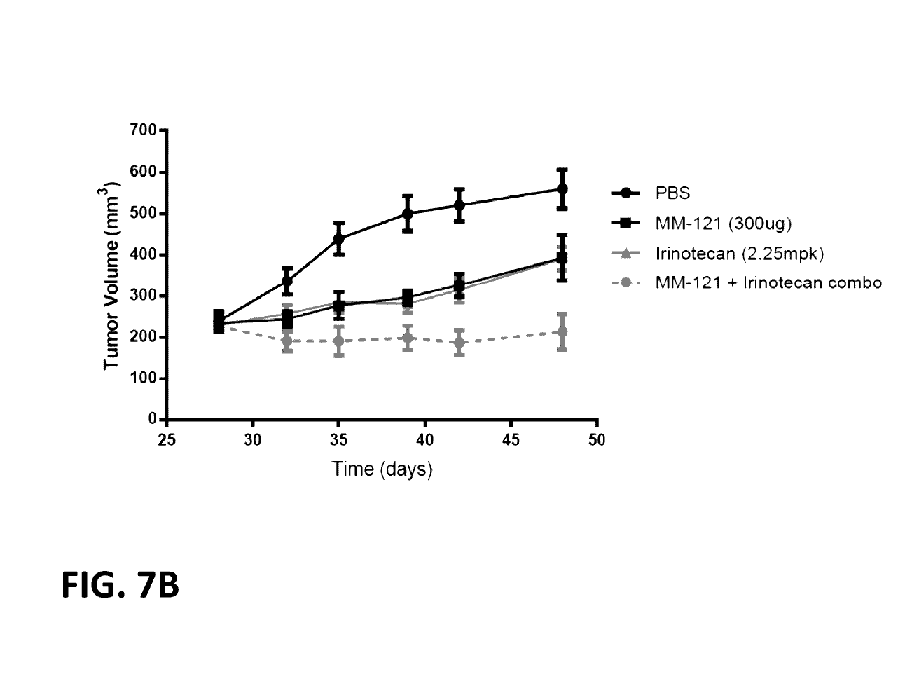

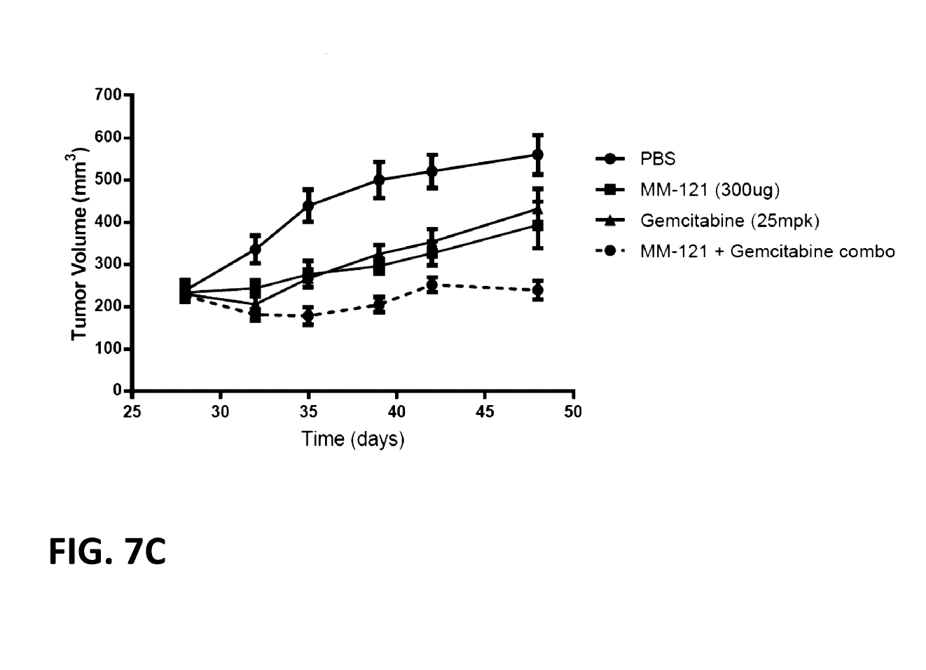

As shown in FIGS. 7A-7C (seribantumab (“MM-121” in the figure) mouse dose; 300 μg Q3D), seribantumab as a single agent significantly suppressed tumor growth in a dose-dependent manner in vivo in this model of ovarian cancer. Moreover, while irinotecan HCl, gemcitabine, and paclitaxel alone each inhibited tumor growth in vivo, combination treatments with seribantumab and paclitaxel (FIG. 7A), irinotecan HCl (FIG. 7B), or gemcitabine (FIG. 7C) exhibited an additive effect on tumor growth inhibition, as compared to tumor growth inhibition observed with each of the individual agents.

### Endnotes

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein. The disclosure of each and every US, international, or other patent or patent application or publication referred to herein is hereby incorporated herein by reference in its entirety.

SEQUENCE SUMMARY

| SEQ ID | | | |
|---|---|---|---|
| NO: | DESIGNATION | | SEQUENCE |
| 1 | Heavy Chain | Human | His Tyr Val Met Ala |
| | CDR1 (CDRH1) | CDRH1 | |
| | of Seribantumab | Protein | |
| 2 | Heavy Chain | Human | Ser Ile Ser Ser Ser Gly Gly Trp Thr Leu |
| | CDR2 (CDRH2) | CDRH2 | Tyr Ala Asp Ser Val Lys Gly |
| | of Seribantumab | Protein | |
| 3 | Heavy Chain | Human | Gly Leu Lys Met Ala Thr Ile Phe Asp Tyr |
| | CDR3 (CDRH3) | CDRH3 | |
| | of Seribantumab | Protein | |
| 4 | Light Chain | Human | Thr Gly Thr Ser Ser Asp Val Gly Ser Tyr |
| | CDR1 (CDRL1) | CDRL1 | Asn Val Val Ser |
| | of Seribantumab | Protein | |
| 5 | Light Chain | Human | Glu Val Ser Gln Arg Pro Ser |
| | CDR2 (CDRL2) | CDRL2 | |
| | of Seribantumab | Protein | |
| 6 | Light Chain | Human | Cys Ser Tyr Ala Gly Ser Ser Ile Phe Val |
| | CDR3 (CDRL3) | CDRL3 | Ile |
| | of Seribantumab | Protein | |
| 7 | Heavy Chain of | Human | 1 EVQLLESGGG LVQPGGSLRL SCAASGFTFS HYVMAWVRQA PGKGLEWVSS |
| | Antibody | Heavy | 51 ISSSGGWTLY ADSVKGRFTI SRDNSKNTLY LQMNSLRAED TAVYYCTRGL |
| | Seribantumab | Chain | 101 KMATIFDYWG QGTLVTVSSA STKGPSVFPL APCSRSTSES TAALGCLVKD |
| | | Protein | 151 YFPEPVTVSW NSGALTSGVH TFPAVLQSSG LYSLSSVVTV PSSNFGTQTY |
| | | | 201 TCNVDHKPSN TKVDKTVERK CCVECPPCPA PPVAGPSVFL FPPKPKDTLM |
| | | | 251 ISRTPEVTCV VVDVSHEDPE VQFNWYVDGV EVHNAKTKPR EEQFNSTFRV |
| | | | 301 VSVLTVVHQD WLNGKEYKCK VSNKGLPAPI EKTISKTKGQ PREPQVYTLP |
| | | | 351 PSREEMTKNQ VSLTCLVKGF YPSDIAVEWE SNGQPENNYK TTPPMLDSDG |
| | | | 401 SFFLYSKLTV DKSRWQQGNV FSCSVMHEAL HNHYTQKSLS LSPGK |
| 8 | Light Chain of | Human | 1 QSALTQPASV SGSPGQSITI SCTGTSSDVG SYNVVSWYQQ HPGKAPKLII |
| | Seribantumab | Light | 51 YEVSQRPSGV SNRFSGSKSG NTASLTISGL QTEDEADYYC CSYAGSSIFV |
| | | Chain | 101 IFGGGTKVTV LGQPKAAPSV TLFPPSSEEL QANKATLVCL VSDFYPGAVT |
| | | Protein | 151 VAWKADGSPV KVGVETTKPS KQSNNKYAAS SYLSLTPEQW KSHRSYSCRV |
| | | | 201 THEGSTVEKT VAPAECS |
| 9 | Heavy Chain | Human | gaggtgcagc tgctggagag cggcggaggg |
| | Variable Region | VH | ctggtccagc caggcggcag cctgaggctg |
| | (VH) of | DNA | tcctgcgccg ccagcggctt caccttcagc |
| | Seribantumab | | cactacgtga tggcctgggt gcggcaggcc |
| | | | ccaggcaagg gcctggaatg ggtgtccagc |
| | | | atcagcagca gcggcggctg gaccctgtac |
| | | | gccgacagcg tgaagggcag gttcaccatc |
| | | | agcagggaca acagcaagaa caccctgtac |
| | | | ctgcagatga acagcctgag ggccgaggac |
| | | | accgccgtgt actactgcac caggggcctg |
| | | | aagatggcca ccatcttcga ctactggggc |
| | | | cagggcaccc tggtgaccgt gagcagc |
| 10 | Heavy Chain | Human | Glu Val Gln Leu Leu Glu Ser Gly Gly Gly |
| | Variable Region | VH | Leu Val Gln Pro Gly Gly Ser Leu Arg Leu |
| | (VH) of | Protein | Ser Cys Ala Ala Ser Gly Phe Thr Phe Ser |
| | Seribantumab | | His Tyr Val Met Ala Trp Val Arg Gln Ala |
| | | | Pro Gly Lys Gly Leu Glu Trp Val Ser Ser |
| | | | Ile Ser Ser Ser Gly Gly Trp Thr Leu Tyr |
| | | | Ala Asp Ser Val Lys Gly Arg Phe Thr Ile |
| | | | Ser Arg Asp Asn Ser Lys Asn Thr Leu Tyr |
| | | | Leu Gln Met Asn Ser Leu Arg Ala Glu Asp |
| | | | Thr Ala Val Tyr Tyr Cys Thr Arg Gly Leu |
| | | | Lys Met Ala Thr Ile Phe Asp Tyr Trp Gly |
| | | | Gln Gly Thr Leu Val Thr Val Ser Ser |
| 11 | Light Chain | Human | cagtccgccc tgacccagcc cgccagcgtg |
| | Variable Region | VL | agcggcagcc caggccagag catcaccatc |
| | (VL) of | DNA | agctgcaccg gcaccagcag cgacgtgggc |
| | Seribantumab | | agctacaacg tggtgtcctg gtatcagcag |
| | | | caccccggca aggcccccaa gctgatcatc |
| | | | tacgaggtgt cccagaggcc cagcggcgtg |
| | | | agcaacaggt tcagcggcag caagagcggc |
| | | | aacaccgcca gcctgaccat cagcggcctg |
| | | | cagaccgagg acgaggccga ctactactgc |
| | | | tgcagctacg ccggcagcag catcttcgtg |
| | | | atcttcggcg gagggaccaa ggtgaccgtc |
| | | | cta |
| 12 | Light Chain | Human | Gln Ser Ala Leu Thr Gln Pro Ala Ser Val |
| | Variable Region | VL | Ser Gly Ser Pro Gly Gln Ser Ile Thr Ile |
| | (VL) of | Protein | Ser Cys Thr Gly Thr Ser Ser Asp Val Gly |
| | Seribantumab | | Ser Tyr Asn Val Val Ser Trp Tyr Gln Gln |
| | | | His Pro Gly Lys Ala Pro Lys Leu Ile Ile |
| | | | Tyr Glu Val Ser Gln Arg Pro Ser Gly Val |
| | | | Ser Asn Arg Phe Ser Gly Ser Lys Ser Gly |
| | | | Asn Thr Ala Ser Leu Thr Ile Ser Gly Leu |
| | | | Gln Thr Glu Asp Glu Ala Asp Tyr Tyr Cys |
| | | | Cys Ser Tyr Ala Gly Ser Ser Ile Phe Val |
| | | | Ile Phe Gly Gly Gly Thr Lys Val Thr Val |
| | | | Leu |
| 13 | Human ErbB3 | Human | Ser Glu Val Gly Asn Ser Gln Ala Val Cys |
| | | Protein | Pro Gly Thr Leu Asn Gly Leu Ser Val Thr |
| | | | Gly Asp Ala Glu Asn Gln Tyr Gln Thr Leu |
| | | | Tyr Lys Leu Tyr Glu Arg Cys Glu Val Val |
| | | | Met Gly Asn Leu Glu Ile Val Leu Thr Gly |
| | | | His Asn Ala Asp Leu Ser Phe Leu Gln Trp |
| | | | Ile Arg Glu Val Thr Gly Tyr Val Leu Val |
| | | | Ala Met Asn Glu Phe Ser Thr Leu Pro Leu |
| | | | Pro Asn Leu Arg Val Val Arg Gly Thr Gln |
| | | | Val Tyr Asp Gly Lys Phe Ala Ile Phe Val |
| | | | Met Leu Asn Tyr Asn Thr Asn Ser Ser His |
| | | | Ala Leu Arg Gln Leu Arg Leu Thr Gln Leu |
| | | | Thr Glu Ile Leu Ser Gly Gly Val Tyr Ile |
| | | | Glu Lys Asn Asp Lys Leu Cys His Met Asp |
| | | | Thr Ile Asp Trp Arg Asp Ile Val Arg Asp |
| | | | Arg Asp Ala Glu Ile Val Val Lys Asp Asn |
| | | | Gly Arg Ser Cys Pro Pro Cys His Glu Val |
| | | | Cys Lys Gly Arg Cys Trp Gly Pro Gly Ser |
| | | | Glu Asp Cys Gln Thr Leu Thr Lys Thr Ile |
| | | | Cys Ala Pro Gln Cys Asn Gly His Cys Phe |
| | | | Gly Pro Asn Pro Asn Gln Cys Cys His Asp |
| | | | Glu Cys Ala Gly Gly Cys Ser Gly Pro Gln |
| | | | Asp Thr Asp Cys Phe Ala Cys Arg His Phe |
| | | | Asn Asp Ser Gly Ala Cys Val Pro Arg Cys |
| | | | Pro Gln Pro Leu Val Tyr Asn Lys Leu Thr |
| | | | Phe Gln Leu Glu Pro Asn Pro His Thr Lys |
| | | | Tyr Gln Tyr Gly Gly Val Cys Val Ala Ser |
| | | | Cys Pro His Asn Phe Val Val Asp Gln Thr |
| | | | Ser Cys Val Arg Ala Cys Pro Pro Asp Lys |
| | | | Met Glu Val Asp Lys Asn Gly Leu Lys Met |
| | | | Cys Glu Pro Cys Gly Gly Leu Cys Pro Lys |
| | | | Ala Cys Glu Gly Thr Gly Ser Gly Ser Arg |
| | | | Phe Gln Thr Val Asp Ser Ser Asn Ile Asp |
| | | | Gly Phe Val Asn Cys Thr Lys Ile Leu Gly |
| | | | Asn Leu Asp Phe Leu Ile Thr Gln Gly Asp |
| | | | Pro Trp His Lys Ile Pro Ala Leu Asp Pro |
| | | | Glu Lys Leu Asn Val Phe Arg Thr Val Arg |
| | | | Glu Ile Thr Gly Tyr Leu Asn Ile Gln Ser |
| | | | Trp Pro Pro His Met His Asn Phe Ser Val |
| | | | Phe Ser Asn Leu Thr Thr Ile Gly Gly Arg |
| | | | Ser Leu Tyr Asn Arg Gly Phe Ser Leu Leu |
| | | | Ile Met Lys Asn Leu Asn Val Thr Ser Leu |
| | | | Gly Phe Arg Ser Leu Lys Glu Ile Ser Ala |
| | | | Gly Arg Ile Tyr Ile Ser Ala Asn Arg Gln |
| | | | Leu Cys Tyr His His Ser Leu Asn Trp Thr |
| | | | Lys Val Leu Arg Gly Pro Thr Glu Glu Arg |
| | | | Leu Asp Ile Lys His Asn Arg Pro Arg Arg |
| | | | Asp Cys Val Ala Glu Gly Lys Val Cys Asp |
| | | | Pro Leu Cys Ser Ser Gly Gly Cys Trp Gly |
| | | | Pro Gly Pro Gly Gln Cys Leu Ser Cys Arg |
| | | | Asn Tyr Ser Arg Gly Gly Val Cys Val Thr |
| | | | His Cys Asn Phe Leu Asn Gly Glu Pro Arg |
| | | | Glu Phe Ala His Glu Ala Glu Cys Phe Ser |
| | | | Cys His Pro Glu Cys Gln Pro Met Glu Gly |
| | | | Thr Ala Thr Cys Asn Gly Ser Gly Ser Asp |
| | | | Thr Cys Ala Gln Cys Ala His Phe Arg Asp |
| | | | Gly Pro His Cys Val Ser Ser Cys Pro His |
| | | | Gly Val Leu Gly Ala Lys Gly Pro Ile Tyr |
| | | | Lys Tyr Pro Asp Val Gln Asn Glu Cys Arg |
| | | | Pro Cys His Glu Asn Cys Thr Gln Gly Cys |
| | | | Lys Gly Pro Glu Leu Gln Asp Cys Leu Gly |
| | | | Gln Thr Leu Val Leu Ile Gly Lys Thr His |
| | | | Leu Thr Met Ala Leu Thr Val Ile Ala Gly |
| | | | Leu Val Val Ile Phe Met Met Leu Gly Gly |
| | | | Thr Phe Leu Tyr Trp Arg Gly Arg Arg Ile |
| | | | Gln Asn Lys Arg Ala Met Arg Arg Tyr Leu |
| | | | Glu Arg Gly Glu Ser Ile Glu Pro Leu Asp |
| | | | Pro Ser Glu Lys Ala Asn Lys Val Leu Ala |
| | | | Arg Ile Phe Lys Glu Thr Glu Leu Arg Ser |
| | | | Leu Lys Val Leu Gly Ser Gly Val Phe Gly |
| | | | Thr Val His Lys Gly Val Trp Ile Pro Glu |
| | | | Gly Glu Ser Ile Lys Ile Pro Val Cys Ile |
| | | | Lys Val Ile Glu Asp Lys Ser Gly Arg Gln |
| | | | Ser Phe Gln Ala Val Thr Asp His Met Leu |
| | | | Ala Ile Gly Ser Leu Asp His Ala His Ile |
| | | | Val Arg Leu Leu Gly Leu Cys Pro Gly Ser |
| | | | Ser Leu Gln Leu Val Thr Gln Tyr Leu Pro |
| | | | Leu Gly Ser Leu Leu Asp His Val Arg Gln |
| | | | His Arg Gly Ala Leu Gly Pro Gln Leu Leu |
| | | | Leu Asn Trp Gly Val Gln Ile Ala Lys Gly |
| | | | Met Tyr Tyr Leu Glu Glu His Gly Met Val |
| | | | His Arg Asn Leu Ala Ala Arg Asn Val Leu |
| | | | Leu Lys Ser Pro Ser Gln Val Gln Val Ala |
| | | | Asp Phe Gly Val Ala Asp Leu Leu Pro Pro |
| | | | Asp Asp Lys Gln Leu Leu Tyr Ser Glu Ala |
| | | | Lys Thr Pro Ile Lys Trp Met Ala Leu Glu |
| | | | Ser Ile His Phe Gly Lys Tyr Thr His Gln |
| | | | Ser Asp Val Trp Ser Tyr Gly Val Thr Val |
| | | | Trp Glu Leu Met Thr Phe Gly Ala Glu Pro |
| | | | Tyr Ala Gly Leu Arg Leu Ala Glu Val Pro |
| | | | Asp Leu Leu Glu Lys Gly Glu Arg Leu Ala |
| | | | Gln Pro Gln Ile Cys Thr Ile Asp Val Tyr |
| | | | Met Val Met Val Lys Cys Trp Met Ile Asp |
| | | | Glu Asn Ile Arg Pro Thr Phe Lys Glu Leu |
| | | | Ala Asn Glu Phe Thr Arg Met Ala Arg Asp |
| | | | Pro Pro Arg Tyr Leu Val Ile Lys Arg Glu |
| | | | Ser Gly Pro Gly Ile Ala Pro Gly Pro Glu |
| | | | Pro His Gly Leu Thr Asn Lys Lys Leu Glu |
| | | | Glu Val Glu Leu Glu Pro Glu Leu Asp Leu |
| | | | Asp Leu Asp Leu Glu Ala Glu Glu Asp Asn |
| | | | Leu Ala Thr Thr Thr Leu Gly Ser Ala Leu |
| | | | Ser Leu Pro Val Gly Thr Leu Asn Arg Pro |
| | | | Arg Gly Ser Gln Ser Leu Leu Ser Pro Ser |
| | | | Ser Gly Tyr Met Pro Met Asn Gln Gly Asn |
| | | | Leu Gly Glu Ser Cys Gln Glu Ser Ala Val |
| | | | Ser Gly Ser Ser Glu Arg Cys Pro Arg Pro |
| | | | Val Ser Leu His Pro Met Pro Arg Gly Cys |
| | | | Leu Ala Ser Glu Ser Ser Glu Gly His Val |
| | | | Thr Gly Ser Glu Ala Glu Leu Gln Glu Lys |
| | | | Val Ser Met Cys Arg Ser Arg Ser Arg Ser |
| | | | Arg Ser Pro Arg Pro Arg Gly Asp Ser Ala |
| | | | Tyr His Ser Gln Arg His Ser Leu Leu Thr |
| | | | Pro Val Thr Pro Leu Ser Pro Pro Gly Leu |
| | | | Glu Glu Glu Asp Val Asn Gly Tyr Val Met |
| | | | Pro Asp Thr His Leu Lys Gly Thr Pro Ser |
| | | | Ser Arg Glu Gly Thr Leu Ser Ser Val Gly |
| | | | Leu Ser Ser Val Leu Gly Thr Glu Glu Glu |
| | | | Asp Glu Asp Glu Glu Tyr Glu Tyr Met Asn |
| | | | Arg Arg Arg Arg His Ser Pro Pro His Pro |
| | | | Pro Arg Pro Ser Ser Leu Glu Glu Leu Gly |
| | | | Tyr Glu Tyr Met Asp Val Gly Ser Asp Leu |
| | | | Ser Ala Ser Leu Gly Ser Thr Gln Ser Cys |
| | | | Pro Leu His Pro Val Pro Ile Met Pro Thr |
| | | | Ala Gly Thr Thr Pro Asp Glu Asp Tyr Glu |
| | | | Tyr Met Asn Arg Gln Arg Asp Gly Gly Gly |
| | | | Pro Gly Gly Asp Tyr Ala Ala Met Gly Ala |
| | | | Cys Pro Ala Ser Glu Gln Gly Tyr Glu Glu |
| | | | Met Arg Ala Phe Gln Gly Pro Gly His Gln |
| | | | Ala Pro His Val His Tyr Ala Arg Leu Lys |
| | | | Thr Leu Arg Ser Leu Glu Ala Thr Asp Ser |
| | | | Ala Phe Asp Asn Pro Asp Tyr Trp His Ser |
| | | | Arg Leu Phe Pro Lys Ala Asn Ala Gln Arg |
| | | | Thr |

We claim:

1. A method of treating a patient having heregulin (HRG) positive non-small cell lung cancer (NSCLC), the method comprising administering to the patient once on day 1 of a 21-day treatment cycle an anti-neoplastic therapy consisting of:
i. a dose of 3000 mg seribantumab; and
ii. a dose of 75 mg/m² docetaxel,
to treat the NSCLC in the patient.

2. The method of claim 1, wherein the cancer is positive for HRG mRNA as measured by RNA in-situ hybridization (RNA-ISH), wherein the HRG RNA-ISH results in a score of ≧1+.

3. The method of claim 1, wherein the cancer is positive for HRG as measured by quantitative RT-PCR.

4. The method of claim 1, wherein the patient has failed at least one systemic therapy for locally advanced and/or metastatic NSCLC.

5. The method of claim 1, wherein the patient has progressed following treatment with no more than three systemic therapies for locally advanced or metastatic disease, one of which systemic therapies comprised a platinum-based regimen.

6. The method of claim 1, wherein docetaxel is co-administered at least 30 minutes before administration of seribantumab.

7. The method of claim 1, wherein the anti-neoplastic therapy is administered intravenously.

8. The method of claim 1, wherein the treatment produces at least one therapeutic effect selected from the group consisting of: reduction in size of a tumor, reduction in metastasis, complete remission, partial remission, stable disease, increase in overall response rate, or a pathologic complete response.

9. The method of claim 1, wherein the NSCLC is EGFR wild-type.

10. The method of claim 1, wherein the NSCLC is a squamous cell carcinoma.

11. A method of treating a patient having HRG positive non-small cell lung cancer (NSCLC, the method comprising administering to the patient once on day 1 of a 21-day treatment cycle an anti-neoplastic therapy consisting of:
i. a dose of 3000 mg seribantumab; and
ii. a dose of 500 mg/m² pemetrexed,
to treat the NSCLC in the patient.

12. The method of claim 10, wherein the tumor is positive for HRG mRNA as measured by RNA in-situ hybridization (RNA-ISH), wherein the HRG RNA-ISH results in a score of ≧1+.

13. The method of claim 11, wherein the cancer is positive for HRG as measured by quantitative RT-PCR.

14. The method of claim 11, wherein the patient has failed at least one systemic therapy for locally advanced and/or metastatic NSCLC.

15. The method claim 11, wherein the patient has progressed following treatment with no more than two systemic therapies for locally advanced or metastatic disease, one of which systemic therapies comprised a platinum-based regimen.

16. The method of claim 11, wherein the pemetrexed is co-administered at least 30 minutes before the administration of seribantumab.

17. The method of claim 11, wherein the treatment produces at least one therapeutic effect selected from the group consisting of: reduction in size of a tumor, reduction in metastasis, complete remission, partial remission, stable disease, increase in overall response rate, or a pathologic complete response.

18. The method of claim 11, wherein the NSCLC is EGFR wild-type.

19. The method of a claim 11, wherein the NSCLC is a squamous cell carcinoma.

20. The method of claim 11, wherein the antineoplastic therapy is administered intravenously.


SEQUENCE LISTING (13 sequences)

SEQ ID NO: 1 - protein, 5 aa, Homo sapiens
HYVMA

SEQ ID NO: 2 - protein, 17 aa, Homo sapiens
SISSSGGWTLYADSVKG

SEQ ID NO: 3 - protein, 10 aa, Homo sapiens
GLKMATIFDY

SEQ ID NO: 4 - protein, 14 aa, Homo sapiens
TGTSSDVGSYNVVS

SEQ ID NO: 5 - protein, 7 aa, Homo sapiens
EVSQRPS

SEQ ID NO: 6 - protein, 11 aa, Homo sapiens
CSYAGSSIFVI

SEQ ID NO: 7 - protein, 445 aa, Homo sapiens
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 8 - protein, 217 aa, Homo sapiens
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 9 - DNA, 357 bp, Homo sapiens
gaggtgcagctgctggagagcggcggagggctggtccagccaggcggcagcctgaggctgtcctgcgccgccagcggcttcaccttcagccactacgtgatggcctgggtgcggcaggccccaggcaagggcctggaatgggtgtccagcatcagcagcagcggcggctggaccctgtacgccgacagcgtgaagggcaggttcaccatcagcagggacaacagcaagaacaccctgtacctgcagatgaacagcctgagggccgaggacaccgccgtgtactactgcaccaggggcctgaagatggccaccatcttcgactactggggccagggcaccctggtgaccgtgagcagc

SEQ ID NO: 10 - protein, 119 aa, Homo sapiens [sequence omitted]

SEQ ID NO: 11 - DNA, 333 bp, Homo sapiens
cagtccgccctgacccagcccgccagcgtgagcggcagcccaggccagagcatcaccatcagctgcaccggcaccagcagcgacgtgggcagctacaacgtggtgtcctggtatcagcagcaccccggcaaggcccccaagctgatcatctacgaggtgtcccagaggcccagcggcgtgagcaacaggttcagcggcagcaagagcggcaacaccgccagcctgaccatcagcggcctgcagaccgaggacgaggccgactactactgctgcagctacgccggcagcagcatcttcgtgatcttcggcggagggaccaaggtgaccgtccta

SEQ ID NO: 12 - protein, 111 aa, Homo sapiens [sequence omitted]

SEQ ID NO: 13 - protein, 1321 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITQGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRSLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT